(19) United States
(12) Patent Application Publication
(10) Pub. No.: US 2005/0227315 A1
(43) Pub. Date: Oct. 13, 2005

(54) Exogenous protein expression system in an avian system

(76) Inventors: Bertrand Pain, Lyon (FR); Jacques Samarut, Villeurbanne (FR); Isabelle Valarche, Nantes (FR); Patrick Champion-Arnaud, Nantes (FR); Andre Sobczyk, Nantes (FR); Ryota Kunita, Kanagawa (JP)

Correspondence Address: Blakely Sokoloff Taylor & Zafman, 12400 Wilshire Boulevard, 7th Floor, Los Angeles, CA, 90025, US

(21) Appl. No.: 10/496,397

(22) PCT Filed: Nov. 21, 2002
(86) PCT No.: PCT/FR02/03992

§ 371 Date: May 21, 2004

(30) Foreign Application Priority Data
Nov. 22, 2001 (FR) 0115111

(51) Int. Cl. C12P 21/06; C12N 5/06; C12N 15/85

(52) U.S. Cl. 435/69.1; 435/455; 435/349; 530/362

(57) ABSTRACT

The invention concerns the use of an avian cell for producing an exogenous protein of interest in an animal belonging to the avian species, said cell being transformed by an expression vector comprising the gene coding for said protein, said cell being introduced in the sub-germinal cavity of an embryo, the blood stream of the embryo or being used as nucleus source for nuclear transfer into an avian egg enucleated or not, or whereof the chromosomes have been destroyed.

This invention relates to the use of an avian cell for the production of an exogenous protein of interest in an animal belonging to the avian species, the said cell being transformed by an expression vector comprising the coding gene for the said protein, the said cell being introduced either into the sub-germinal cavity of an embryo, the blood circulation of the embryo or acting as nucleus source for the nuclear transfer in an enucleated or non-enucleated ovocyte, or for which the chromosomes have been destroyed.

Traditionally, the first pharmaceutical constituents were obtained by extraction from tissues of different vegetable, animal or even human origins. Three main factors have contributed to the placement of substitutes for this extraction technique. Rarefaction of tissues due to the increase in demand, risks inherent to animal and human derivatives have increased, with the appearance or identification of new pathologies, and particularly viral and non-conventional pathologies. Finally and particularly, the search for and the use of discoveries in different branches of biology (biochemistry, molecular biology, etc.) have help to discover and therefore satisfy the ever-increasing demand for new molecules.

In order to satisfy the demand for therapeutic products, molecule expression systems such as proteins for therapeutic use, have been associated with an active biological component (bacteria, yeast, higher eukaryotic cell, etc.) and a genetic element that provides the necessary support for production. The various genetic supports used are usually either simple expression vectors optimised for expression but for which integration remains uncertain, or viral or retroviral systems. Viral or retroviral systems cannot be used to control the expression location or level.

These production methods are currently undergoing a new digital and qualitative revolution. Thus, within a few years, due to continuous and fast progress made in genomic and molecular biology, the number of molecules with one or several therapeutic and medical capacities has significantly increased. For example, antibodies for therapeutic purposes already represent 50% of these molecules. Moreover, the complexity of these recombining molecules has increased. Starting from simple peptides or small cytokines that are easily produced in bacteria, complex molecules are gradually reached with a biological activity that is strictly dependent on their secondary structure, their folding and complex post-translational modifications. Consequently, a simple production means in bacteria is no longer sufficient.

More sophisticated techniques have subsequently emerged, including in vitro culture of eukaryotic cells, yeast and insect cells. Even more recently, progress made in vegetable and animal transgenesis has been used to produce these complex molecules either in plant leaves or seeds (tobacco, corn, etc.) or in physiological animal liquids such as animal milk (goat, cow, sow, rabbit, etc.).

Furthermore, the correlation between the screening efficiency of a given locus and its transcription level, particularly at cells targeted by the vector including stem cells, is not absolute. Thus, these methods always present problems such as the production cost, the quantity of the products obtained and technical difficulties when complex molecules are to be produced.

These problems can be solved according to the invention by producing therapeutic molecules targeting the expression in specific tissues, particularly in the egg and particularly in egg white, using animals modified with stem cells, themselves modified by recombination vectors.

The different steps necessary for use of the invention involve firstly the construction of vectors, that target the locus chosen in an avian cell, and particularly a stem cell, obtaining recombined cells that include this vector in the chosen locus and generation of modified animals with these cells.

One of the advantages of this complete system is the controlled nature of the expression of the protein of interest. The replacement substitution action of an endogenous avian locus by an exogenous gene or gene fragment of interest is a means of obtaining a production of the exogenous protein introduced instead of the endogenous locus. For example, by introducing the gene to be produced by the complete described mechanism into the locus of ovalbumin, the product of this gene will be produced according to the perfectly known expression profile of ovalbumin. Thus, the invention provides a solution to problems raised by random integration of a heterologous sequence, the expression of which may depend on its chromatinian environment.

Moreover, and unlike the methods mentioned above, the avian production system according to the invention provides a means of obtaining larger quantities of active substances at lower cost, considering that they may be easily isolated and purified.

A recombination process in a line of lymphoid cells (the DT40 line) was made possible using a recombination process by means of a particular mutation (the mutation affects the expression level of protein Rad54) (Kim et al., 1990; Baba et al., 1988; Buerstedde et al., 1990; Buerstedde and Takeda, 1991; Bezzubova et al., 1993, Bezzubova et al., 1997). In this publication, the authors clearly state that they were unable to make the homologous recombination with ES cells.

Unlike this information, the applicant has demonstrated that a targeted homologous recombination process enables an expression of an exogenous molecule of interest in avian systems and that this same process can be controlled experimentally in different types of avian cells including lines, primary cells and stem cells and embryo stem cells.

Therefore, the purpose of the invention is:
homologous recombination vectors to target and substitute for egg proteins and particularly egg white proteins, either totally or partially, by molecules of interest,
obtaining specifically modified avian stem cells at loci targeted by the said vectors,
use of the avian cells specifically modified by these vectors for an in vitro expression of molecules of interest,
obtaining and production of large quantities of molecules of interest by the animals obtained with these avian cells specifically modified by the said recombination vectors.
The production of molecules of interest in organs or tissues of the said avian species animals enabling extraction, isolation and simple and economically advantageous purification.

Therefore, this invention as a whole provides a means of offering an innovative system for the production of molecules of interest according to a perfectly predictable mode since it is controlled both in space and in time, providing solutions to problems that exist in the state of the art.

### DESCRIPTION

Thus, the first aspect to the invention relates to the use of an avian cell for the production of an exogenous protein of interest in an animal belonging to the avian species, characterised in that the said cell is transformed by an expression vector comprising the coding gene for the said protein, the said cell being introduced either into the sub-germinal cavity of an embryo, the blood circulation of the embryo or acting as a nucleus source for the nuclear transfer in a enucleated or non-enucleated ovocyte, or for which the chromosomes have been destroyed.

An avian cell denotes a precursor cell, a somatic cell, a stem cell, an embryonic stem cell (ES), a pluripotent cell, a totipotent cell, a germinal cell, a germinal stem cell (EG), a primordial germinal cell (PGC), a cellular clone of the above mentioned cells, a line produced from the above mentioned cells or an embryoid body obtained from the above-mentioned cells.

The different cells mentioned above are defined in more detail below:
A precursor cell means any cell of a partially differentiated adult or embryonic tissue that is capable of dividing itself and producing differentiated cells.
A somatic cell means any cell of an embryo or an adult, other than a germinal cell.
An embryonic stem cell (ES) means any non-differentiated cell deriving from an embryo and which has the potential to result in a very wide variety of specialised cells.
A stem cell means any cell with a capacity to divide itself in vitro in culture during infinite time periods and capable of resulting in different types of specialised cells.
A pluripotent cell means a single cell that has the capacity to differentiate itself into cells in the three embryonic sheets.
A totipotent cell means a cell with an unlimited capacity for proliferation, state limited to an egg cell.
A germinal cell means spermatozoids and the ovocyte, or their precursors capable of resulting in a spermatozoid and an ovocyte. All other cells are somatic cells.
Germinal stem cells (EG) means cells located in a specific part of the embryo called the genital ridges, becoming the gonads in the adult organism. These cells normally develop into mature gametes.
A cellular clone means all cells genetically identical to the cell from which they are derived by division.
An embryoid body means any aggregate of cells derived from embryonic stem cells maintained in culture. Embryoid bodies do not participate in the normal development of an embryo and can only be observed in vitro.
A line means any cellular culture capable of proliferating indefinitely while keeping the same phenotypical characteristics.
   I—Different Vectors can be used for Transformation of the said Cells:

The vector used for this purpose enables a tissue specific expression, particularly in the oviduct, the liver, blood, bone marrow and lymphoid organs, regardless of whether it is a recombination vector or a simple expression vector.

I.1—Recombination Vectors

The homologous recombination vector targets an endogenous locus to express a gene of interest instead of this endogenous locus. For chromatin, the homologous recombination is a process that consists of partially or completely replacing part of a gene by a construction that partly or completely repeats some sequences identical to this same gene. The identical parts enable an exchange of DNA added into the endogenous locus and introduction of the modified parts into the environment considered and therefore respected by the targeted locus. The result is an insertion of modifications made by some parts of the construction into the targeted locus. The different parts of the construction provide a means of controlling the expression of the introduced cassettes. These cassettes may be dependent on their own promoter, or they may be dependent on the transcriptional activity of the targeted locus when the cassette is made dependent on the internal promoter.

The recombination reaction may be divided into three main steps:
The first step is matching of homologous regions, leading to a “three-strand” transient structure of the DNA.
The second step is production of an independent double crossing over in homology regions, leading to the formation of a hybrid molecule between the targeted chromosome and the introduced vector.
The third step is integration of this vector as a stable genetic element of the targeted chromosome, the modification then being propagated like all genetic material with cellular divisions.

Any element present in the system or added into it in a stable or transient manner that controls, modifies or influences these various mechanisms will make an essential contribution to the recombination process and therefore will influence the integration efficiency of the vector.

In the context of the invention, the recombination vector that produces molecules in the avian system, particularly in a chicken, is a replacement substitution vector rather than a simple destruction vector. These complex vectors are the result of an ordered and successive association in a classical cloning plasmid (pBSK, pUC, etc) of at least one of the following elements:
A fragment of genomic DNA with variable size, taken from the input side of the ATG (translation start site) of the targeted gene. This element forms the arm 5′.
A cDNA or a part of the gene(s) of interest, placed directly under the dependence of the ATG of the endogenous targeted gene. This association of fragments is usually made directly by fusion of targeted locus onto the ATG with the second amino acid of the protein. Furthermore, to enable export directly into the egg of the protein of interest, a nucleotidic sequence specifying a signal peptide must be placed on the upstream side of the cDNA of interest and on the downstream side of the signals from the targeted gene. It is added either by the fragment of interest, or by an additional exogenous nucleotidic sequence.

A cDNA means all or part of a coding sequence of a nucleic acid for a protein of interest.

A protein of interest means a protein, a fragment of a protein or a peptide that has a therapeutic advantage for man or animal, but also any proteic form that can provide a benefit to man or an animal in terms of his or its physical status, his or its health, his or its behaviour or vitality, concerning particularly a protein or a peptide of endogenous interest and a protein or a peptide of exogenous interest defined below:

A protein of endogenous interest means any protein present in the system, particularly the egg, present at variable levels but detectable in a normal physiological situation.

A protein of exogenous interest means any protein of exogenous origin to the system, not identified in the system, particularly the egg, in a normal physiological state. Its presence is the direct or indirect result of the genetic and/or biochemical modification induced by the vector into the system.
A resistance cassette (called a selection cassette positive to an exogenous selection system) generally composed of a promoter, a cDNA that adds resistance to the exogenous selection, and a poly A sequence enabling stopping the transcription of this independent transcriptional element. The promoter may be from different origins.
A fragment of genomic DNA of variable size taken from the downstream side of these different elements and therefore the ATG of the targeted gene. In general, the size of this fragment is not the same as the fragment of genomic DNA located at 5′. This element forms the arm 3′.
An additional cassette called the negative selection cassette may be incorporated into this vector. It is usually integrated on the downstream side of the genomic arm 3′. This cassette is located at 3′ of the homology arm 3′, and does not persist in the genome of the targeted cell if integration took place according to the correct scheme. Only events not correctly recombined are sensitive to this action.
At least one unique linearisation site on the vector, recommended by experience. This step appears necessary for efficient introduction of this vector into the genetic heritage of the targeted cell.
Different elements particularly enabling conditional modifications may be inserted at different locations of the construction.

The main characteristics of a recombination vector in mammal systems as they have been described up to now depend on the succession of the different elements within the plasmid and in the positioning of the genomic arms (origin of DNA, size of homologous genomic elements, relative asymmetry, etc). In most cases, the recombination frequency observed in vitro is dependent on the targeted locus and therefore the targeted chromatinian environment (Ramirez-Solis et al., 1993; Hasty et al., 1995; Hasty et al., 1191). This environment is closely related to the physiology of the cell. It appears that the selection pressure applied to transfected cells enables a number of integrations, most of which are non-homologous compared with the number of homologous integrations. The screening process, for which the principle and examples are described in the text, provides a means of distinguishing between the two event types. Knowing, evaluating and controlling the proportion of homologous integrations is essential for a satisfactory expression strategy. The repeatability of the recombination between added fragments and original fragments must also be as good as possible. This appears to be the case at least for mouse ES cells and human fibroblasts (Zheng et al., 1991, Lederman et al., 2000; Templeton, 2000).

Moreover, the composition based on a locus alone can have a strong influence on recombination efficiencies, particularly due to the strong influence of the many CpG islands (Yanez and Porter, 2000). Other cryptic elements (poison sequence) may also modify the efficiency of the homologous recombination process (replication initiation sites, repeated sequences, etc.). Moreover, the homologous recombination frequency would appear to be very dependent on the target cell, with special situations such as for the line of DT40 lymphoid cells in the chicken, which has impressive recombination ratios (>10%). This specific feature is related to over-expression of the Rad54 molecule, undoubtedly related to a mutation not yet identified, that participates in the control of the regulation of this gene (Bezzubova et al., 1993; Bezzubova et al., 1994; Bezzubova et al., 1997). This observation can result in an increase in the recombination frequency.

In the context of the invention, the efficiency of homologous recombination has been demonstrated in avian embryonic cells, and some other primary avian cells that have been established in line.

In a complementary embodiment, Rad54 or a protein of the rad family can be over-expressed in order to improve the efficiency of the recombination process.

Length of Homologous Fragments

A few studies carried out in embryonic stem cells of the mouse on different loci indicate that a minimum length for homology arms 5′ and 3′ is necessary to achieve a recombination at the locus with a satisfactory frequency (Hasty et al., 1991, Thompson et al., 1989; Thomas and Capecchi, 1987). If a minimum size of 250-500 bp appears sufficient to achieve a recombination event of one of the two sides, the key parameter would tend to be more the total homology length than the corresponding size of the homology arms (Elliott et al., 2001; Philips and Calos, 1999; Fujitani et al., 1995). This limit would be at least 5 to 6 kb. Extrapolation of the results from the very complete data obtained from bacteria, might suggest that the limiting factor in the recombination efficiency would not be in searching for homologies between donor DNA fragments added by the vector and receptor DNA parts of the locus to be targeted (Yancey-Wrona and Caerini-Otero, 1995), but rather in the exchange system between these fragments that follows matching. However, extreme caution is necessary in interpreting these results and extrapolating them in the eukariote system, which may be sensitive to the presence of hot spots facilitating recombination in special loci.

Immunoglobulin loci would appear to be very special in this respect. Homologous integration events of a vector with homology only in the arm at 3′ have been observed (Berinstein et al., 1992). Moreover, recombinations in immunoglobulin loci appear particularly as a result of the activity of sites recognised by recombinases specific to these genes. Some interference with the equipment of these cells and the recombination process may be observed.

Thus in one particular aspect, the vector is a homologous recombination vector with homology arms 5′ and 3′ with the sequences of a given locus. The targeted locus may for example be selected from among the locus of ovalbumin, ovomucoids, conalbumin and lysozyme.

Consequently, the vector comprises heterologous segments:

This presence of an important homologous part is particularly necessary when heterologous segments are important (Kumar and Simons, 1993). Thus, the isogenic approach (Te Riele et al., 1992) is reinforced by the observation of a drop in the homologous recombination frequency in the case of an interruption in a long fragment of homologies by variable stretches related to the presence of polymorphism of a locus (Lukacsovich and Waldman, 1999). But the symmetric presence of heterologous segments located on the input side of specific homology elements can also reinforce homologous integration levels of the vector. This presence would protect homologies from degradations induced by exonucleases.

Induction of a Targeted Cut

Any targeted induction (for example by a meganuclease) of a double strand cut, a specific and rare cut in the DNA of the targeted cell, increases recombination frequencies. It would appear that this induction triggers the repair system and facilitates incorporation of the exogenous DNA (Bibikova et al., 2001; Sargent et al., 2000; Donoho et al., 1998; Sargent et al., 1997; Brenneman et al., 1996; Hasty et al., 1992b). This approach is used particularly by introducing a specific site recognised by a meganuclease, particularly of the I-Scel or other type in order to give priority to recombination events (Sarig et al., 2000; Cohen-Tanoudji et al., 1998; Robine et al., 1998; Jasin et al., 1996).

Linearisation of a Vector

Comparative tests were carried out between linear and circular vectors. In the murine system, it appears that linearisation is a necessary prerequisite to increase frequencies. Other delivery actions sometimes have an influence (Yanez and Porter, 1999).

Conditional Structure of Expression Vectors

The various elements currently present in vectors are active constitutively once the homologous recombination event has been carried out. The expression of an exogenous protein in the egg can lead to toxicity both in the tissue in which production will take place, and possibly in the egg, the final accumulation location. Therefore, it must be possible to induce and decide upon this accumulation both in time and in space.

One possible approach is to use inducible and conditional systems.

An inducible system may for example mean the system called Tet off/tet on. This system is composed of two elements: the donor and the operator. The donor system itself includes a genetic construction derived from a system of resistance to tetracycline, identified in a bacterial transposon Tn10. In the absence of tetracycline, the repressor protein TetT, constitutively expressed, blocks transcription of the gene enabling resistance to tetracycline. In the presence of tetracycline, the repressor can no longer bond to the control sequences. This system was used advantageously by fusing this tetR sequence with the transactivating domain of the viral protein VP16 (Grossens and Bujard, 1992) patent U.S. Pat. No. 5,589,362. The result is tTa proteins (standing for Tetracycline controlled Trans Activator) capable of acting on the modified TetO (for Tet Operator) promoter itself. In the case of vectors described in the invention, a first “donor” vector (a simple vector or for example targeting a specific locus of the oviduct tissue by recombination) is added into the stem cell and elements sensitive to the action of tetracycline are placed within the recombination construction that targets the locus of ovalbumin, for example. These elements are inserted into a region of the ovalbumin promoter to control the expressed protein level transcriptionally.

Other inducible systems include fusion of these different players (VP16, Gal4, etc.) with proteic control sequences as some receptors to nuclear hormones, more or less modified to specifically respond to the presence of hormone analogues (Muted ER binding the tamoxifen (Metzger et al., 1995; Indra et al., 1999), the Receptor ecdysone, the PR binding the RU486, etc.).

For example, a conditional system means systems implementing some enzymes like some recombinases. These enzymes recognise particular and specific small sequences (IoxP sequences for CRE enzyme, FLP sequence for FRT recombinase, etc.) that are then inserted in the construction to be controlled. The general action mode is to delete elements located between these sequences when the recombinase is active. This recombinase is introduced by different methods (transient transfection, integrated stable expression, etc.) and its expression controls the excision reaction at the introduced sequences.

The conditional system is made inducible by combining the presence of these recombinases with inducible systems through nuclear hormone receptors. Thus, for example, the recombinase CRE can be fused with the muted version of ER, sensitive to tamoxifen. This system was used advantageously in the murine system (Metzger and Chambon, 2001; Vallier et al., 2001). Other systems such as FLT-EcDR (Sawicki et al., 1998) have been described for the murine system.

Basic Elements of Constructions

The origin of basic elements of vectors is variable. The skeletons of the main plasmids are obtained from commercial sources. They can be used for easy propagation of constructions that they carry into a well controlled bacterial environment, namely the E. coli environment, even if different stems are used. Thus, the pBSK, pMCS5, pCI Neo plasmids are used to be a general basis for many intermediate or terminal vectors.

Positive selection cassettes are composed of:
a promoter originating from different sources, viral for example such as the CMV promoter (cyomegalovirus), the promoter of the SV40 virus, the LTR of the RSV or non-viral sources such as promoters of ubiquitous genes (such as the b-actine promoter of chicken) or promoters of genes specifically expressed at some development phases or gene promoters specifically expressed in some tissues. A special case is to use a specific promoter for the state of stem cells.
a coding gene for the “positive” resistance to an antibiotic, for example such as the resistance gene to neomycin, hygromycin, puromycin, phleomycin, zeomycin, blasticidin, viomycin, and also the DHFR (dihydrofolate reductase) gene providing resistance to methotrexate, the HPRT (Hypoxanthine phosphoribosyl transferase) gene responsible for the transformation of specific bases present in the HAT selection medium (aminopterin, hypoxanthine, thymidine) and other genes for detoxification with respect to some drugs.

Negative selection cassettes are composed of:
a promoter as described above
a gene for transformation of a substrate present in the culture medium into a toxic substance for the cell that expresses the gene. These molecules include detoxification genes of diphteric toxin (DTA) (Yagi et al., 1998; Yanagawa et al., 1999), the kinase thymidine gene of the Herpes virus (HSV TK) sensitive to the presence of gancyclovir or FIAU. The HPRT gene may also be used as a negative selection by addition of 6-thioguanine (6TG) into the medium.
and for all positive and negative selections, a poly A transcription termination sequence from different origins, the most classical being derived from SV40 poly A, or a eukaryotic gene poly A (bovine growth hormone, rabbit b-globin, etc.).
A special case is making the positive selection cassette directly dependent on the targeted endogenous locus. This approach requires functionality and therefore transcriptional activation of the targeted locus before it can be used and therefore the selection of clones after transfection must be linked to a particular state of differentiation or non-differentiation. These examples are not limitative.

Thus, the homologous recombination vector according to the invention includes chaining of at least one element from among the following sequence, in a plasmidic base:
h) a fragment of genomic DNA containing the homology arm 5′ of the targeted fused gene fused with,
h) a nucleotidic sequence signalling secretion fused with,
h) a short intronic nucleotidic sequence fused with,
h) the nucleotidic sequence coding for the protein of interest fused with
h) a poly A termination sequence of the transcription fused with
h) a positive selection cassette including a promoter, a resistance gene to a selection agent and a poly A transcription termination sequence, the said cassette possibly being fused with
h) a fragment of genomic DNA containing the homology arm 3′ of the targeted gene,
h) a negative selection cassette including a promoter, a gene for transformation of a substrate present in the culture medium into a toxic substance for the cell that expresses the gene and a polyA transcription termination sequence.
Promoter means any nucleotidic sequence capable of initiating and directing the transcription of a gene on the downstream side of this sequence.

This vector may also include d) the coding sequence for the exogenous protein fused at its end 5′ with c) a short intronic sequence particularly including the sequence SEQ ID No. 1 itself fused with e) a secretion signal peptide sequence, particularly the coding sequence for the peptide signal of the lysozyme including sequence SEQ ID No. 2.

The vector according to the invention may also include d) the coding sequence for the exogenous protein fused at its end 3′ with a poly A sequence.

The vector may also include at least one IRES sequence fused with at least two coding sequences for the exogenous protein of interest or at least one IRES sequence fused with at least two coding sequences for different chains forming a protein of interest, particularly heavy and light chains of an antibody of any nature whatsoever, particularly a monoclonal antibody, a fab fragment.

The IRESs chosen will be IRESs from group I or group II, particularly the V130 sequences (Patents U.S. Pat. No. 5,925,656, FR 95/00894), Idemfix and Zam (WO 99/29844), (Leblanc et al., 1997; 1999).

1.2—Simple Expression Vector

These vectors are characterised by the successive association of a promoter, a cDNA or part of a gene, and a poly A sequence that stops the transcription. After transfection of these vectors in eukaryotic cells, these vectors have the advantage and disadvantage of being integrated into the genome at random. The efficiency is often good and a large number of integration sites can be obtained. However, the random aspect of this insertion makes the expression starting from the promoter carried by the vector dependent on the environment of the insertion site (methylation, imprinting, enhancer, silencer, etc.).

Thus, promoters that can be used in the context of the invention capable of overcoming these difficulties and used for the purposes of a specific tissue expression, include (non exhaustively) the promoter of the lysozyme gene (from 2500 to 100 bp), and also the promoter of the ovalbumin gene, in long forms (from 5 to 1 kb) containing positive and negative or short regions (from 1000 to 100 bp), knowing that a minimum promoter of about 100 bp can be specifically activated by different hormones (Monroe and Sanders, 2000).

Example 1 below illustrates the possibilities of obtaining clones from stem cells with such simple expression vectors.

Therefore, the useful vector within the context of the invention may be an expression vector comprising the coding sequence for the protein of interest fused with at least one element taken from among:
a) a promoter, particularly selected from among promoters of ovalbumin genes, ovomucoids, conalbumin and lysozyme,
b) a peptide signal sequence
c) a poly A transcription termination nucleotidic sequence.

This expression vector and the recombination vector may include an IRES sequence fused with at least two sequences coding for the same protein of interest or for different coding sequences.

The above-mentioned vectors transform an avian cell like that defined above.

Advantageously, the said cell is a primary embryonic avian cell, an embryonic avian stem cell, particularly embryonic stem cells derived from creating a culture of blastoderms.
a blastoderm means an avian embryo at a stage preceding gastrulation and comprising a simple cellular organization with at least two layers of cells separated by a cavity.

The avian embryonic cell according to the invention has a positive phosphatase alkaline phenotype, particularly a positive phosphatase alkaline phenotype of an embryonic stem cell.

Avian embryonic cells, embryo stem cells and embryonic germinal cells are characterised in that they react specifically with at least one antibody chosen from among ECMA-7, SSEA-1,SSEA-3, TEC-01, EMA-1 and EMA-6.

The cell according to the invention may also be a primary avian cell of a phenotype defined particularly for a primary fibroblast, an epithelial cell, and an endothelial cell.

In a more particular embodiment, the cell is an avian cell derived from a primary embryonic cell and spontaneously established in culture or using different immortalising agents, particularly cells derived from induced embryo stem cells to be differentiated under the action of different induction agents and particularly retinoic acid, dimethylsulfoxide, TPA or specific culture conditions, particularly by the formation of embryoid bodies (see definition above).

Alternately, the cell is an avian cell established in line, particularly LMH hepatic cells, HD11 monocyte cells and QT6 fibroblast cells.

As mentioned above, the said cell may be transformed with an expression vector expressing a protein in the Rad family, and particularly the Rad54 protein.

Thus, in a second aspect, the invention relates to a process for obtaining an avian cell modified by one of the vectors defined above.

This process may include the following steps:
a) introduction of the vector defined according to one of the above claims into an avian embryonic cell by a transfection method, particularly using a liposome, a polycation or by electroporation
b) selection of cells by the addition of a selection agent into the culture medium, particularly geneticine within a concentration range from 100 to 500 μg/ml
c) screening of resistant clones and amplification.

Preferably, this selection is done for at least 2 to 10 days, and particularly 2, 3, 4, 5 or 6 days.

Advantageously, the invention relates to a process for obtaining an avian cell modified by one of the vectors defined above including the following steps:
a) introduction of the vector defined according to one of the above claims into an avian embryonic cell by a transfection method, particularly using a liposome, a polycation or by electroporation
b) selection of cells by the addition of a selection agent into the culture medium, particularly geneticine within a concentration range from 100 to 500 μg/ml
c) obtaining genetically stable recombined clones with a morphology and characteristics similar to parental cells (endogenous phosphatase alkaline ratio, reactivity to surface antibodies, and telomerase activity)
d) screening of resistant clones and amplification of the resulting correctly recombined resistant cells.

In one special aspect, the recombination vector targets the lysozyme locus.

Another purpose of the invention is a process mentioned above characterised in that the supernatant fluid of the culture of recombined cells contains the exogenous protein of interest, particularly after induction of the clone using different inductors, and particularly retinoic acid, dimethylsulfoxide, TPA or specific culture conditions, particularly by the formation of embryoid bodies.

In this process, the two alleles of the targeted locus are preferably modified.

Furthermore, cells are embryonic avian primary cells, embryonic avian stem cells, particularly embryonic stem cells derived from putting blastoderms into culture. These cells have a positive phosphatase alkaline phenotype, particularly a positive phosphatase alkaline phenotype of an embryonic stem cell.

Avian embryonic cells according to the process are characterised in that they react specifically with at least one antibody selected from among ECMA-7, SSEA-1, SSEA-3, TEC-01, EMA-1 and EMA-6. The cells obtained according to the process according to the invention include particularly a epithelial cell, or an endothelial cell, in addition to primary phenotype avian cells defined in particular for a primary fibroblast.

The invention also relates to a process defined above in which the cells are avian cells derived from a primary embryonic cell and established in line using different immortalising agents, particularly cells derived from induced embryonic stem cells to be differentiated under the action of different induction agents, and particularly retinoic acid, dymethylsulfoxide, TPA or specific culture conditions, particularly by the formation of embryoid bodies.

Alternately, the cells are avian cells established in line, particularly LMH hepatic cells, HD11 monocyte cells and QT6 fibroblast cells.

The said cells defined above may also be transformed with an expression vector expressing a protein in the Rad family, particularly the Rad54 protein.

With this process, the medium used may include anti-retinoic acid antibodies (ARMA) and a cytokine chosen from among the group composed of LIF, IL-11, IL-6 and different mixes of them.

The medium used may also include different factors, particularly SCF, IGF-1, bFGF, CNTF and Oncostatine.

A third aspect of the invention relates to a process for obtaining an animal belonging to the avian species capable of expressing an exogenous protein of interest, characterised in that it comprises the following steps:
a) obtaining avian cells modified by the process defined above
b) introduction of the cell obtained in step a) into the sub-germinal cavity of an embryo, into the blood circulation or by nuclear transfer of the nucleus of the said cell into an enucleated or non-enucleated ovocyte, and
c) incubation of the embryo obtained in step b).
Nuclear transfer means transfer of a nucleus of a cell in an egg or ovocyte from which the nucleus was or was not withdrawn, or in which the chromosomes were destroyed or altered by gamma or UV irradiation or any other appropriate means.

Preferably, the vector used in step a) enables a tissue specific expression, particularly in the oviduct, the liver, blood, bone marrow or lymphoid organs.
Bone marrow means the tissue that fills the cavity of most bones and that contains haematopoietic stem cells, starting from which all red and white blood cells are derived.

The invention also relates to the above mentioned process to obtain an animal belonging to the avian species with a tissue-specific expression of an exogenous protein of interest, characterised in that the vector is a homologous recombination vector possessing arms 5′ and 3′ of homology to sequences of a given locus, and particularly a locus selected from the locus of ovalbumin, ovomucoids, conalbumin and lysozyme, among the different constituent elements necessary for its operation.

This type of vector may include the coding sequence for the exogenous protein fused with at least one element selected from among an intronic sequence, a secretion signal peptide sequence, particularly the lysozyme signal peptide comprising the sequence SEQ ID No. 2, a poly A sequence, an IRES and a promoter, chosen particularly from among promoters of ovalbumin, ovomucoid, conalbumin and lysozyme genes.

Step b) in the process for obtaining an animal belonging to the avian species with a tissue-specific expression of an exogenous protein of interest may also include transformation of avian cells with a vector expressing a protein in the Rad family, particular Rad54.

In one additional aspect, the invention relates to a process for production of a protein of interest including extraction of the exogenous protein expressed in the tissues of an animal obtained from the process mentioned above. In this process, the protein is preferably extracted from blood, or egg yolk or white.

Alternately, the process for production of a protein of interest may consist of extracting the exogenous protein expressed in the supernatant fluid of cells derived from the process according to the invention.

The invention also relates to an animal belonging to the avian species that can be obtained starting from the process described above, characterised in that it expresses an exogenous protein in a specific tissue, for example in the liver, blood, bone marrow, lymphoid organs or the oviduct.

In another aspect, the invention relates to an egg that could be obtained from an animal described above, characterised in that part of these components, particularly ovalbumin, ovomucoids, conalbumin and lysozyme are partially or completely replaced by an exogenous protein of interest, selected particularly from among peptides with a therapeutic interest, interleukines, cytokines, hormones and antibodies.
Antibodies refer to a protein with a specific affinity for an antigen comprising polyclonal, monoclonal antibodies and their Fab fragment.

The egg according to the invention may include a proportion of exogenous protein between a few mg (1 to 10 mg) and 500 mg of dry materials replacing all or part of at least one endogenous protein, particularly chosen from among ovalbumin, ovomucoids, conalbumin, lysozyme and avidine.

Some non-limitative examples of embodiments of the invention are given below.

Abbreviations

cDNA: complementary DNA
CMV: Cytomegalovirus
DNA: Deoxyribonucleic Acid
ER: Estrogen Receptor
FGF: Fibroblast Growth Factor
GFP: Green Fluorescent Protein
GR: Glucocorticoid Receptor
HRE: Hormone Responsive Element
IGF-1: Insulin Growth Factor 1
LPS: Lipopolysaccharides
LTR: Long Terminal Repeat
MAR: Matrix Attachment Region
MCSF: Macrophage Colony Stimulating Factor
NRE: Negative Response Element
PR: Progesterone Receptor
RAR: Retinoic Acid Receptor
RNA: Ribonucleic Acid
RSV: Rous Sarcoma Virus
RXR: Retinoid X Receptor
SCF: Stem Cell Growth factor
SDRE: Steroid Dependent Regulatory Element
TPB: Tryptose Broth Phosphate
TPA: TriPhrobol Ester
TR: Thyroid hormone Receptor
VDR: Vitamin D Receptor.

### EXAMPLE 1

### The Egg as a Production System

The expression of a protein of interest in a physiological liquid of an animal, for example a chicken, particularly in the egg and particularly in the egg white, appears achievable using the different molecular tools including expression vectors. The general principle of the invention is to make an exogenous molecule of interest be expressed directly in the egg without changing either of the proteins already present in the egg, or replacing an endogenous molecule of a fraction of this endogenous molecule.

1.1—Egg White Proteins

Egg is a particularly suitable medium for expression of exogenous molecules. The egg white is a complex medium, and the biochemical composition of this medium is fairly well characterised (see table 1 below).

TABLE 1
average content (in g) for a 60 g egg

| | Full egg | white | yolk |
|---|---|---|---|
| Water | 40 | 31 | 9 |
| Dry material | 20 | 4 | 10 |
| Proteins | 6.9 | 3.6 | 3.2 |
| Lipids | 6.4 | <0.2 | 6.4 |
| Carbohydrates | 0.2 | 0.2 | <0.1 |

According to Sauveur, 1988 mentioned op

The egg white is poor in lipids (0.02%), in inorganic ions and carbohydrates (0.5% including free glucose) and is composed mainly of water (88%) and proteins in solution (11.5%). Egg white proteins are generally well characterised as a whole, although their exact number is still subject to variation (Stevens, 1991; Lu-Chan and Nakai, 1989; Sauveur, 1988). It is generally agreed that about 40 different proteins have been identified. These include five proteins accounting for the majority (ovalbumin, conalbumin, ovomucoid, ovomucin a and b and lysozyme), alone accounting for nearly 83-84% of all proteins in the white. Therefore, considering this composition and the average weight of dry material equal to about 6-7 g, therefore these majority proteins account for the equivalent of about 4.8-5.6 g. Minor proteins, although significantly detectable, account for 5 to 6% of the total proteins. These minority components include avidine, well known in the diagnostic world for its very high affinity and very strong specificity with regard to biotin. The other components are identified in smaller proportions, variable from one preparation to another and are often badly characterised both biochemically and molecularly. The number of studies concerning them is not very large, which is natural considering their low representativeness. The physicochemical properties of proteins forming the majority proteins in egg white are presented (see Table 2 below).

TABLE 2
Protein composition of a hen egg white

| | % of | | | % | | | |
|---|---|---|---|---|---|---|---|
| Protein | protein | MW | pI | carb | Function | AA | S—S |
| Ovalbumin | 54 | 45.0 | 4.5 | 3.5 | Structural | 385 | 1 |
| Conalbumin | 12-13 | 77.7 | 6.0 | 2.6 | Iron | 686 | 15 |
| | | | | | transport | | |
| Ovomucoid | 11 | 28.0 | 4.1 | 16 | Proteinase | 185 | 9 |
| | | | | | inhibitor | | |
| Ovomucin a | 1.5-3.5 | 210 | 4.5 | 13 | Structural | 1276 | |
| Ovomucin b | | 720 | | 58 | | 246 | |
| Lysozyme | 3.5 | 14.3 | 10.7 | 0 | Proteolytic | 129 | 4 |
| | | | | | enzyme | | |
| Ovoinhibitor | 0.5-1.5 | 49.0 | 5.1 | 5-9.6 | Proteinase | | 21 |
| | | | | | inhibitor | | |
| OvoGlycoprotein | 0.5-1.0 | 24.4 | 3.9 | | ? | | |
| Ovoflavoprotein | 0.8 | 32.0 | 4.0 | 11 | Riboflavin | 234 | 9 |
| | | | | | transport | | |
| Ovostatin | 0.5 | 780 | 4.9 | 5.8 | Proteinase | 2762 | |
| | | | | | inhibitor | | |
| OvoGlobulin | >1 | 47 | 4.9 | ? | ? | 102 | |
| G2 | | | | | | | |
| OvoGlobulin | >1 | 50 | 4.8 | ? | ? | 103 | |
| G3 | | | | | | | |
| Cystatin | 0.05 | 12.7 | 5.1 | 0 | Proteinase | 124 | 2 |
| | | | | | inhibitor | | |
| Avidin | 0.05 | 68.3 | 10 | 7 | Biotin | 4 × 129 | 4 × 1 |
| | | | | | transport | | |
| Thiamin | <0.05 | 38 | | 0 | Thiamin | | |
| binding | | | | | transport | | |
| protein | | | | | | | |
| Other | 9 | | | | | | |
| proteins | | | | | | | |

According to Stevens (1991), Comp. Bioch. Physiol 100b 1-9 and Li-Chan and Nakai (1989), Critical review in Poultry Biology 2, 21-58.

All proteins in the egg white are generated by cells in the oviduct, at the magnum. Different cell types have been found and some specialisation between cells is observed among the cells responsible for secretion. The epithelial calciform cells (also called mucus cells) are specialised in the production of avidine and ovomucine, while tubular gland cells preferentially secrete lysozyme and ovalbumin. The distribution of these different cell types is variable in the epithelium that secretes magnum, but no regionalisation is observed (Sauveur, 1988).

Egg white proteins are synthesized continuously in the cells. The glandular and calciform epithelial cells “Istore” proteins that are discharged around the yolk during transit of the magnum within a few hours (about 3h30). The mechanical deformation induces this secretion and this very fast deposit (Sauveur 1988).

Thus, it appears essential that all molecular and physical regulation signals should be maintained, so that secretion of exogenous proteins in the egg white can take place correctly.

In a chimeric animal, the necessary prerequisite for any production of an exogenous molecule of interest is the presence of the genetic modification in the oviduct and in the cells of the magnum in particular. The preferred approach for the use of embryonic stem cells appears to enable this mosaicism. In terms of establishing an animal breed, this contribution is transmitted automatically in the heterozygote state and then the homozygote state by appropriate crossings, since it is integrated in the genome.

1.2—Components of the Yolk

The egg yolk consists essentially of an accumulation of lipids (table 1 above) in the form of lipoproteins. It is the result of a close association of two majority proteins, namely vitelline and vitellinine with phospholipids and triglycerides. The other components (cholesterol, vitamins and liposoluble pigments) are minority. Unlike proteins in the white, all proteins and components of the yolk are synthesised by the liver and are transported by blood circulation and accumulate in the yolk during the development of follicles (Sauveur, 1988; Nau, 1987). The yolk also contains a non-negligible proportion of immunoglobulins that are accumulated directly during production of this vitellus. The presence of these immunoglobulins from maternal origin, also provides some immune protection during the first days, or even longer, of the life of the chicken. Studies are now being started on the transport mechanism for these immunoglobulins and their accumulation in the egg. The purification process of these molecules IgY specific to the bird (equivalent to the IgG of mammals) is well understood. This property can be used to obtain a relatively high quantity of avian immunoglobulins (sometimes of the order of 10 mg per egg) or even to encourage accumulation by a targeted immunisation of the laying hen. It has also been shown that human immunoglobulins can also accumulate in the yolk, by a mechanism similar to the endogenous immunoglobulins (Mohammed et al., 1998). A specific expression strategy may be developed within this objective of making the hen produce particular immunoglobulins.

### EXAMPLE 2

### Description of Molecular Loci and their Transcriptional Regulation

Most genes that specify the majority proteins of the egg white have been studied at the molecular level. The coding phases are usually identified and most sequences of corresponding cDNAs are located in data banks. Genomically, progress has been slower. The structure of loci in most majority molecules has been published completely or partially. However, published sequences may prove to be incomplete (see Table 3 below).

TABLE 3
Identified sequences of proteins in the egg white

| | cDNA | gene | gene (partial) |
|---|---|---|---|
| Ovalbumin | V00383 | J00895 | |
| Ovalbumin X | | | J00917′ ex1, J00918 |
| | | | ex5, J00919 ex6, |
| | | | J00920 ex7 |
| Ovalbumin Y | | J00922 | |
| Conalbumin | X02009 | Y00407 | |
| Ovomucoid | J00902 | | J00897 (5′), J00898 |
| | | | (3′ coding), J00899 |
| | | | (3′ non cod.) |
| Ovomucin | AB046524 | | |
| Lysozyme | X61198 | X98408 | J00882 5′ ex1 |
| | | X00589 | J00883 ex2 |
| | | X00591 | J00884 ex3 |
| | | X05461 | J00885 ex4 3′ |
| | | X05463 | |
| | | X05462 | |
| Ovoinhibitor | | | M15962 ex1, M16127 |
| (16 exons) | | | ex2, M16128 ex3 |
| | | | M16129 ex4, M16130 ex5 |
| | | | M16131 ex6 |
| | | | M16132 ex7, M16133 |
| | | | ex8, M16134 ex9 |
| | | | M16135 ex10, M16136 |
| | | | ex11 |
| | | | M16137 ex12 |
| | | | M16138 ex13, M16139 |
| | | | ex14 |
| | | | M16140 ex15 |
| | | | M16141 ex16 |
| Ovostatin | X78801 | | |
| Cystatin | J05077 | M95725 | X62413 (synthetic mut |
| | | | V55D) |
| | | | X62411 (synthetic mut |
| | | | Q53N |
| | | | S56A) |
| | | | X62412 (synthetic mut |
| | | | R52L Q53E) |
| | | | X62409 (synthetic mut |
| | | | Q53N) |
| | | | X62408 (synthetic mut |
| | | | Q53E) |
| | | | X62407 (synthetic mut |
| | | | L54M G57A) |
| | | | X62410 (synthetic mut |
| | | | Q53N G57C) |
| | | | X62406 (synthetic mut |
| | | | G57A) |
| | | | X14685 (1-23 SIM I29 |
| | | | L89) |
| Avidin (Acd) | X05343 | AJ311647 | |
| Avr1 | | AJ311647 | Z21611 |
| Avr2 | | AJ311648 | Z21554 |
| Avr3 | | | Z21612 |
| Avr4 | | | Z22883 |
| Avr5 | | | Z22882 |
| Avr6 | | AJ237658 | |
| Avr7 | | AJ237659 | |

2.1—The Ovalbumin Locus

The locus of ovalbumin with a size of 100 kb belongs to the multigenic family of serpins and contains 3 related genes, ova, ovaX and ovaY. They are all expressed in the oviduct of a laying hen (LeMeur et al., 1981; Baldacci et al., 1981). The locus of ovalbumin has been known structurally since 1979 through electronic microscopy studies (Gannon et al., 1979) and is located on chromosome 2 (Dominguez-Steglich et al., 1992). The first complete sequence that was published in 1978 is for ovalbumin. Its pre-mRNA has a size of 7564 bp and it comprises 7 exons (McReynolds et al., 1978). The spliced messenger size is 1872 bp (Woo et al., 1981).

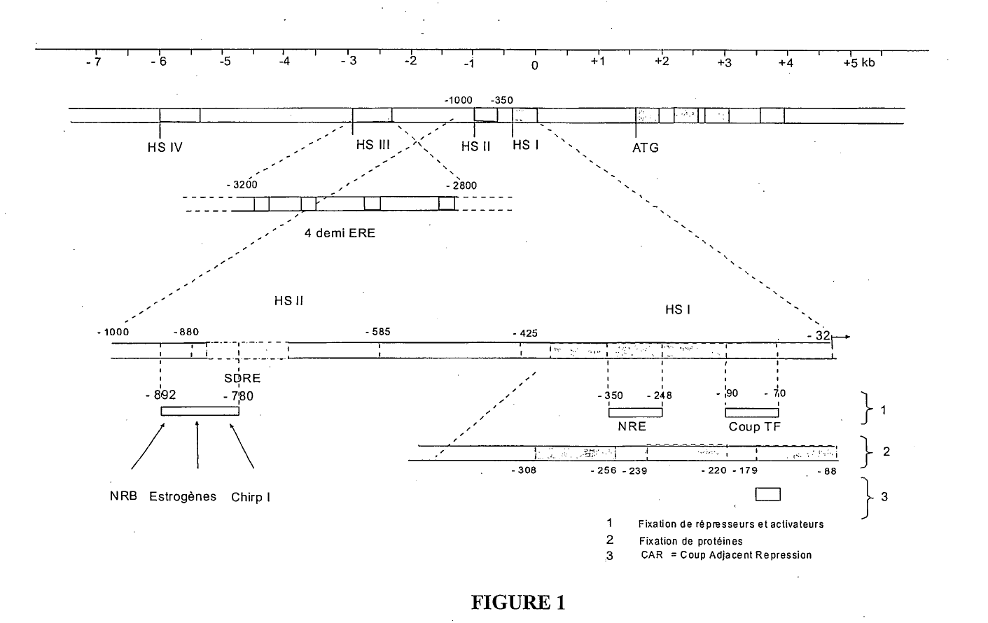

Transcription of the coding gene for ovalbumin—(in the same way as for conalbumin—see 2.3) is controlled by steroidal hormones. Thus, the addition of estrogens stimulates the production of ovalbumin by 20 times and the production of conalbumin by 2.5 times (N'guyen et al., 1979). Before hormonal stimulation, the transcripts of ovalbumin cannot be detected in oviduct cells, while they represent more than 50% of the total transcripts of a cell, in proportion to the dose of estrogen, after maximum stimulation (Dean et al., 1983). If the transcription rate of conalbumin is directly proportional to the quantity of estrogen receptors in the nucleus, the transcription rate of ovalbumin indicates that there are several attachment sites for estrogen receptors in the promoter (Palmiter et al., 1981). The nucleosomic structure of ovalbumin and conalbumin genes is modified during transcriptional activation. The genomic fragments associate with the nuclear matrix and have four major regions of hypersensitivity to DNAse, following the treatment of hormones (Ciejek et al., 1983). These regions are localised in region 5′ of the promoter and 3′ of the last exon at the polyadenylation site (Bellard et al., 1982; Bellard et al., 1986) (FIG. 1: Regulation elements of the Ovalbumin gene). In association with estrogens, different steroid hormones (progesterone, glucocorticoids or androgens) and different factors (Insulin, IGF-1, cAMP, etc.) are also involved in the transcriptional control. In particular, all these factors increase the half-life of the mRNA of the ovalbumin, this half-life increasing from 6 to 24 h after the addition of some of these factors (Arao et al., 1996; Kida et al., 1995; Evans and McKnight, 1984). These factors also increase messenger translation ratios (Skafar et al., 1985; Skoufos and Sanders, 1992).

The results of different work carried out with the ovalbumin promoter have identified different target nucleotidic regions of the action of these hormones and factors (Sensenbaugh and Sanders, 1999). Work on successive deletions at the promoter and transfection experiments of the different constructions show that the promoter is optimally activated in hen primary oviduct cells, regardless of whether or not estrogens are present. Therefore the presence of activators and specific tissue repressors is essential for the expression of this gene (Dierich et al., 1987). FIG. 1 illustrates the main identified regulation sites.

Thus, in sequence, there is an HSIV region at about −6000 bp relative to the transcription initiation site, that does not have any estrogen response elements. An HS III region located between −3200/−2800 bp relative to the transcription initiation site, includes the major regulation sites by estrogens. Deletion of this region abolishes most of the HSIII response, mediated by four repeated half ERE sequences, separated from each other by about a hundred nucleotides. These elements cooperate with the TATA box site located nearby. Each site weakly fixes a receptor, but cooperation of the different sites results in a strong inducible character, in a minimum or heterologous promoter environment. This same region shows elements inhibiting expression of the gene, active elements in non-productive cells, but in a situation outside the oviduct (Kato et al., 1992; Muramatsu et al., 1995).

Region HS II comprises the SDRE (Steroid Dependent Regulatory Element) located between −800 to −585 bp relative to site (+1) at which the transcription starts. This region has homologies with the promoter of the NF-kappa B gene, suggesting controls by common factors (Schweers and Sanders, 1991). Several factors in the W-H proteins family, Chirp-I, Chirp II, Chirp III (CHicken ovalbumin Induced Regulatory Protein I) proteins are also fixed on the SDRE only in the presence of estrogens and glucocorticoids (Dean et al., 1996; Dean et al., 1998; Dean et al., 2001). Once again, to show the complexity of the regulations involved, the presence of this SDRE region alone within a heterologous promoter does not lead to a dependent response of steroid hormones. However, this region becomes dependent on these hormones, if it is associated with the HSI region.

This HS I region located between −350 to −32, is the NRE (negative Response Element) region that controls the main negative regulation of the promoter. NRE represses expression of the gene in the absence of steroidal hormones. The presence of NRE alone or SDRE alone is not sufficient to regulate an exogenous gene. These sequences must cooperate to “induce derepression” (Hasty et al., 1994; Schweers et al., 1990; Sanders and McKnigth, 1988). But the vision is simplistic, since this HSI region is actually composed of a genuine mosaic of sites providing positive and negative regulation of the transcription of the gene. Three domains can be identified, in terms of the influence of their effects. Domain I: region −239 to −220 is a silencer type sequence. The three zones have the affinity for specific proteic complexes of oviduct cells, but the most important are regions −280 to −252 and −134 to −88. This region is marked with the CAR pattern (COUP Adjacent Repression). The region called NRE is actually more complex than when it was initially described (Haecker et al., 1995). A positive regulation site that fixes the delta EF1 factor (Estrogen responsive transcription factor) has also been identified. Thus, interaction sites (−197 to −95) of progesterone and estrogens partially overlap (Dean et al., 1983; Dean et al., 1984). Additional deletions on the output side of the position of −95 bp make the ratios of observed transcripts drop dramatically (Knoll et al., 1983). Apart from estrogen receptors, one of the important regulating factors is the COUP-TF (Chicken Ovalbumin Upstream Promoter Transcription Factor) orphan receptor (Sawaya et al., 1996; Robinson et al., 1999). This factor is expressed ubiquitously in all tissues. Identified by co-purification with the estrogen receptor, it has an affinity with a region located between −90 and −70 bp (Wang et al., 1987; Hwung et al., 1988a; Hwung et al., 1988b; Monroe and Sanders, 2000). The formation of receptor-receptor complexes between COUP-TF and the different receptors to nuclear hormones (VDR, ER, TR, GR, PR, RXR, HNF-4, etc.) in the different HREs, including ERE, encourages attachment to these regulation elements. COUP-TF regulates the action of ER by direct contacts with the DNA, but also through direct protein—proteins between interactions receptors (Klinge et al., 1997). The action modes of COUP-TF and the different related receptors (COUP-TFII or ARPI) are complex (Kimura et al., 1993) that may be positive or negative for regulation of the transcription according to the proteic partners involved. The proteins involved non exhaustively include ear2, (Pereira et al., 1995); N—COR (Nuclear Repressor Coreceptor) and SMRT (Silencing Mediator for retinoic acid and Thyroid hormone Receptor), (Shibata et al., 1997).

2.2—The Lysozyme Locus

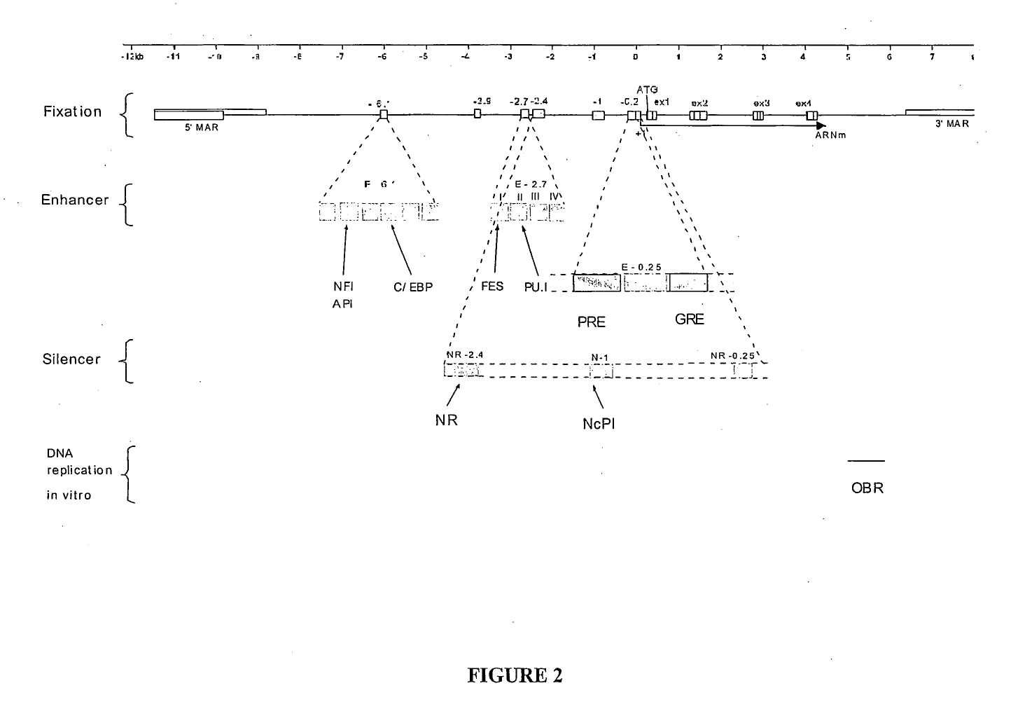

The size of the lysozyme locus is more restricted than the ovalbumin locus. It is estimated at about 40 kb (Sippel et al., 1978; Short et al., 1996). Two MAR (Matrix Attachment Region) units located at −11.1 kb/−8.85 and +1.3 kb to +5.0 kb, located relative to the transcription initiation site (+1) more precisely delimit this locus to 22 kb. These MAR sequences are composed of several latching sites to the proteic matrix of the nuclear membrane (Loc and Stratling, 1988; Phi-van and Stratling, 1996; Phi-van, 1996). The addition of MAR sequences to heterologous genes increases their transcription (Stief et al., 1989; Phi-van et al., 1990). In addition to the presence of MARs, there is at least a DNA replication source in the lysozyme locus, which contains several replication initiation forks (Bonifer et al., 1997; Phi-van et al., 1998; Phi-van and Stratling, 1999). This point also appears to make the lysozyme locus quite specific in terms of cloning, since some parts are quite difficult to sub-clone in cloning plasmids. The lysozyme gene in the chicken comprises 4 exons and has a large homology with human and murine lysozyme genes. Several secondary sites initiating the transcription have been identified, usually located in repeated sequences distributed around the entire locus (Von Kries and Stratling, 1988). Regulating sequences have been identified from −14 kb to +6 kb, around the entire locus. The lysozyme gene is expressed constitutively in mature macrophages, cells in which the lysozyme is used as a differentiation marker, while its expression is placed under the control of steroidal hormones in oviduct cells (Short et al., 1996; Fritton et al., 1987; Jantzen et al., 1986; Fritton et al., 1983). These expression specificities are dependent on different sequences, the main sites of which are marked in FIG. 2: Regulation elements of the lysozyme gene.

A distinction is made between two categories of regulating sequences: so-called positive regulation sequences or “enhancers” and so-called negative regulation sequences or “silencers”. These sequences have been defined as being “hypersensitive” regions following analysis of the electrophoretic profiles of DNA, profiles obtained after treatment with DNAse I.

The main enhancer and silencer sites are the E-6.1 kb, E-2.7 kb, E-0.2 kb and N-2.4 kb, N-1.0 kb and N-0.25 kb regions respectively. Some are specific to myeloid cells (E-6.1 kb, E-2.7 kb, E-0.2 kb for mature macrophages and N-2.4 kb in fibroblasts and immature macrophages), (Steiner et al., 1987; Huber et al., 1995). Element E-6.1 kb is responsible for most of this specificity in cells in the active oviduct. The sequential activation of all these different sites is also a specific cell and tissue regulation element (Regenhard et al., 2001; Kontaraki et al., 2000). In more detail, the enhancer E-2.7 kb is composed of four regions (I to IV). Region I fixes the FEF factor (c-fes Expression Factor), region II contains a recognition site of the PU.1 factor in the EST family, and is specific to macrophages (Ahne and Stratling, 1994). All these different regulation elements overlap partially, at least in the chicken gene, but not in the mouse gene, an ancestral duplication in the mouse having separated the gene and its regulation elements. Other elements have been localised, particularly attachment sites of ubiquitous transcription factors such as NF-1, but also PA1 located at −0.2 kb and −6.1 kb, site on which a 157 bp region makes the expression specificity in macrophages (Goethe et al., 1994; Theisen et al., 1986; Grewal et al., 1992; Nowock and Sippel, 1982). Interestingly, the v-myc oncogene (for example present in HD11 cells) and inhibitor of the differentiation of monocytes in macrophages, blocks the transcription by inhibiting attachment of the C/EBP factor (Mink et al., 1996).

The elements (HRE) responsible for sensitivity of the transcription to steroidal hormones (PRE for progesterone, and GRE glucocorticoids) are present in the region located at −0.2 kb from the site (+1) at which the promoter transcription is initiated (Renkawitz et al., 1982; Renkawitz et al., 1984a; Renkawitz et al., 1984b, Hecht et al., 1988). More particularly, two sites between −220 and −140 bp and between −80 to −50 bp account for most of the regulation (Altschmied et al., 1989; Dolle and Stratling, 1990; Von der Ahe et al., 1986).

A large amount of the regulation is controlled by “silencers”. Two of these silencers (N-1.0 kb and N-0.25 kb) inhibit the transcription, even of heterologous promoters (Baniahmad et al., 1987; Faust et al., 1999). The third (N-2.4 kb) is recognised by thyroid hormone receptors (TR) and by its oncogenic homologue v-erbA, and by the NeP1 regulator (negative Protein 1) also called CTCF (CCCTC-binding factor, (Burcin et al, 1997; Darling et al, 1993; Kohne et al., 1993; Bhat et al, 1994). NeP1 is fixed on part F1 from 50 bp of N-2.4, while TR is fixed mainly on part F2 of this element. Homodimeric or heterodimeric associations with the other nuclear receptors (RAR, RXR) modify the transcription level of this gene (Baniahmad et al., 1990; Arnold et al., 1996). The state of phosphorylation of the TR receptor is also an important element. Finally, the HNF-1 alpha factor (Hepatic nuclear Factor-I) that is expressed in oviduct cells performs a role in regulation of the lysozyme gene. The gene promoter contains two regions recognised by this protein HNF-I. This regulation of the expression of the lysozyme gene by NHF-I appears to have been lost at the phylogenetic level between birds and mammals (Grajer et al, 1993).

2.3—The Conalbumin Locus

The conalbumin gene, also called ovotransferrine, was firstly identified in man. It extends over at least 33.5 kbp and includes 17 exons. The chicken gene is also organised in 17 exons and 16 introns but only over 10.5 kb. The size of the messenger is 2376 bp (Cochet et al., 1979; Jeltsch and Chambon, 1982; Jeltsch et al., 1987; Schaeffer et al., 1987). Very few studies have been made on regulation of the expression of this gene. The regulation of the messenger stability is unusual. The molecule that resembles the transferrines family, fixes iron in ionic form and regulates the stability and the transcription level of its own messenger. Although large numbers of studies have been carried out on human transferrine and its receptor (CD71), there is little documentation about avian forms.

### EXAMPLE 3

### Facilitating External Elements

In eukaryotes, homologous recombination is a natural event that occurs particularly at the time of meiosis, in a simplistic manner to avoid mixing of genes and alleles. The role of homologous recombination is much more difficult to interpret during a mitotic division. Nevertheless, homologous and non-homologous recombination phenomena appear crucial as soon the genetic material is damaged. Many situations in the life of a cell and an organism thus expose the genetic material. For example, a sudden replication defect, a radicalar ionisation related to radiation, an oxidising stress, the action of endonucleases or topoisomerases, the mechanical stress related to mitotic segregation, can induce accidental double strand cuts. Although yeast gives priority to homologous repair mechanisms, the upper eukaryotic cell appears to give priority to non-homologous end joining of these cuts, even if the homologous repair is also observed. But in all cases, since genetic integrity is a prerequisite to any division, in order to handle this critical damaged material situation, the cell starts up a complete set of genes responsible for repairing these cuts. Initially described in the yeast system, the Rad (rad51, rad52, rad53 and rad54) genes (Takata et al., 2000; Morrison and Takeda, 2000; Bell et al., 1999; Shinohara et al., 1997; Bezzubova et al., 1997; Ivanov and Haber, 1997; Porter et al., 1996) involved in repair by homologous recombination have also been identified in higher organisms. The Rad51 and Rad54 homologues of the chicken were cloned. The DNA and the protein have strong homologies with yeast, mouse and human sequences (Essers et al., 1997; Dronkert et al., 2000). Action similarities are then observed, but differences are also observed particularly concerning action of Rad 51 (Bezzubova et al., 1993). Similarly, the human homologues of the ku70 and ku86 genes of yeast are involved in non-homologous repairs, and in recombination phenomena observed in rearrangements of immunoglobulin loci. Relations between these different players are also starting to become closer and they demonstrate the formation of proteic complexes for which the regulations themselves are complex (Morrison et al., 2000; Morrison et al., 1999; Yamaguchi-Iwai et al., 1999; Takata et al., 1998; Yamaguchi-Iwai et al., 1998). Thus, a non-exhaustive list of proteins that facilitate recombination (recombinase) may be given, from the simple bacterian protein RecA (Shcherbakova et al., 2000), to members of the Rad family, and also to proteins such as BraC1 (Moynahan et al., 1999).

In one approach that would facilitate homologous recombination events, the action of Rad 54 is evaluated at the time that the homologous recombination vector is added. Thus, two approaches are possible. The first consists of making a co-transfection of an expression vector of one of the proteins of the Rad family, particularly Rad 54 with the homologous recombination vector. The second consists of transfecting the cells with a conditional expression vector of one of the proteins of the Rad family, particularly Rad 54. In this case, the cells are firstly selected, stabilised and established so that they can once again be transfected with a homologous recombination vector, and then induced to express a maximum amount of the protein of the Rad family. The action of Rad54 tested in terms of the transfection efficiency is given in table 4 below:

TABLE 4
Influence of the expression of Rad 54 on the
number of clones in transfection

| | Condition | | | | | |
|---|---|---|---|---|---|---|
| | 1 | 2 | 3 | 4 | 5 | Control |
| ppreOva RH | 5 | 5 | 5 | 5 | 5 | 0 |
| (μg) | | | | | | |
| pMCS5 (μg) | 5 | 4 | 3 | 2 | 0 | 10 |
| pRad54 (μg) | 0 | 1 | 2 | 3 | 5 | 0 |
| Fugene | 30 | 30 | 30 | 30 | 30 | 30 |
| DMEM | 970 | 970 | 970 | 970 | 970 | 970 |
| No. of clones | | | | | | |
| Box 1 | 344 | 304 | 172 | 248 | 292 | 0 |
| Box 2 | 128 | 324 | 212 | 264 | 300 | 0 |
| Average | 236 | 314 | 193 | 256 | 296 | 0 |

The pRad54 expression plasmid (expression plasmid of protein RAD54 controlled by the CMV promoter) is co-transfected in the presence of the pOvaRH plasmid, a recombination plasmid not containing any cDNA of interest, in order to test the importance of one of the proteins in the Rad family. The total quantity of plasmid is 5 μg of pOvaRH in the presence of variable quantities of pRad54. Since the molar complement is added by a “carrier” plasmid, the pMCS5 originating from a commercial source is approximately the same size as the Rad54 pCMV. The S86N stem cells are seeded at 1×10⁶ cells per box in a proliferation medium in the presence of feeder deactivated by irradiation. The transfection mix is added and the selection with neomycin is applied for about 7 days. Clones are then counted after attachment with methanol and Wright-Giemsa colouring. In conclusion, the presence of Rad54 does not appear to significantly modify the efficiency in obtaining clones.

### EXAMPLE 4

### Choice of the Peptide Signal

One of the approaches chosen to produce exogenous proteins is to accumulate them in the egg. The substitution replacement strategy used within the context of the invention makes an active system useful for accumulation of the exogenous protein in the egg white (but a passive system may also be useful for specific proteins that have this capacity). Thus, the coding phases are placed under the dependence of the endogenous promoter of the endogenous targeted gene and are fused with a reference peptide signal. The peptide signal function is to achieve translocation of the protein towards the outside of the cell through the different component elements of the cell secretion system (reticulum, Golgi, etc.). The signal peptides are intended to be cleaved during the protein maturing process. The protein then accumulates in the physiological liquid and in particular in the egg white. Either signal peptides of endogenous proteins or signal peptides of other molecules are used, which are sequences known to be correctly treated. In particular, the lysozyme signal peptide is chosen in preference since its structure is well known and the 18 first amino acids are cleaved when the mature protein is exported in the egg white. Moreover, this signal peptide belongs to a protein naturally accumulated in the egg white.

The other signal peptides used include interleukin peptides, some membrane receptor peptides, growth factor peptides secreted by different cell types, and peptides of other proteins known to be secreted in different biological systems. This list is not limitative.

### EXAMPLE 5

### Construction of Recombination Vectors

5.1 Obtaining Intermediate Elements
pCMV Neo polyA cassette

The commercial plasmid pCI Neo (Promega, Madison, US) is opened by HindIII digestion and is ligated to itself intra molecularly to give the pCI Neo plasmid (A HindIII) that makes the phosphotransferase neomycin gene directly dependent on the CMV promoter. The original polyA is kept.
preparation of cDNAs of interest

cDNAs of interest are prepared by PCR amplification with
a trace nucleotide Sma-3′incDNA S containing the SmaI site, part 3′ of the artificial intron of the pCINeo commercial vector and the beginning of the specific sequence of cDNA in 5′, having omitted the cDNA ATG, in order to directly connect the read frame of the cDNA of interest in phase with the read frame of ovalbumin or lysozyme.
A cDNA-SmaI As trace nucleotide containing the SmaI site and region 3′ of the cDNA of interest, possibly by omitting an endogenous polyA cDNA and the trace nucleotide 3′ containing the specific sequences of the cDNA and the SmaI site.

The amplified PCR fragment is hydrolysed by SmaI (provided that this site is not present in the cDNA) and cloned in SmaI in a pMCS5 vector (Mobitech, Germany) in which a 277 bp polyA sequence was firstly sub-cloned in XbaI/XhoI. This polyA sequence itself is derived from a hydrolysis of the pIRESHygro vector (Clontech) by XbaI/NhoI. The orientation of the fragment is then controlled by simple digestion with the card provided containing the cDNA of interest.

If an SmaI restriction site is present in the cDNA of interest, the amplified PCR fragment is subjected to the action of the polymerase T4 DNA to make the ends blunt and is directly cloned in a pMCS5 vector open in SmaI (blunt end).

5.2 Genomic Fragment Obtainment

The genomic DNA of 6 to 9 day old embryos from different strains including the CNR (Red Bare Neck) strain and the S86N strain, is extracted either according to a traditional method with a K Proteinase SDS lysis buffer, followed by extractions with phenol and chlorofrome phenol, or using a preparation kit (Promega or Quiagen kit, for example). The different protocols give the DNA yields and qualities compatible with their respective utilization for the preparation of a genomic DNA bank and PCR fragment obtainment by direct amplification.

In the former case, the genomic DNA is partially digested by the Sau3Al enzyme and the generated ends filled by the Klenow polymerase in presence of two of the four nucleotides necessary for the synthesis. The result is partial filling, to prevent the formation of concatemer inserts. The partially filled inserts are inserted by ligation into the λ-GEM-12 vector. The vectors resulting from this operation are packed in phage capsids. As an example, 3 independent isolates were generated and their titer determined, each isolate titer at about 3×10⁶ pfu/ml. The quality of the bank was verified by studying several inserts taken at random. With an ovalbumin probe, obtained by genomic PCR with oligonucleotides chosen from among the published sequence (Table 3), phage screening makes it possible to obtain inserts, sub-cloned in pBS SK+ plasmid vectors to produce the plasmid #72. These inserts, submitted to a restriction mapping, (including EcoRI and BamHI) were then sequenced. Comparison between the published gene (ref. J00895 and Table No. 3) and the two 5′ and 3′ arms of the preRH vector together with the clone #72 fragments show that the homologies are extremely high at the nucleotides level. Only two segments located at the level of introns 6 and introns 7 show low variations spread over several tens of nucleotides (result not shown). This comparison demonstrates the high preservation of this gene among the analysed strains.

In the second case, different oligonucleotides obtained from the published sequence (Table 3 above) are used to obtain, the fragments giving the arms 5′ and 3′ of the vectors, by genomic PCR.

For the 5′ arm of the pOvaRH vector, the OvaL 2995 S and OvaL 80 AS oligonucleotides located respectively at −2995 bp and at +80 bp respectively relative to the ATG of the ovalbumin gene amplify a region of 3075 bp which, submitted to hydrolysis by NeoI, release a fragment of 2860 bp. In parallel, beginning with the sequence specifying the peptide signal of the lysozyme, a fragment named lyso 1-18-intron is prepared by PCR amplification on the locus of the lysozyme with Lyso 1-18 and GE-IN-AS oligonucleotides. The latter oligonucleotide contains the 30 bp of the artificial intron of the pCINeo plasmid. The two fragments (Ova5′ and Lysol-18 intron) are hydrolysed independently by NeoI, purified and then ligated together, and the amplification on the ligation product with the OvaL 2995 S and GE-IN-AS oligonucleotides gives the Ova5′ Lyso 1-18 fragment of 3050 bp. This PCR product is ligated with EcoRI adaptors, allowing this ensemble to be cloned either in the pBSK vector at the EcoRI site or in the pMCS5 blunt end digested by EcoRV.

A final hydrolysis of this Ova5′Lysol-18 fragment by Bst981 e Ascl releases the cleaved plasmid and a small fragment of 220 bp upstream of the ova5′ fragment. This small deletion makes it possible to design two oligonucleotides Ova5′.1S and Ova5′.2S used later for screening recombination events.

The cleaved plasmid is submitted to the action of Klenov polymerase in order to render the blunt ends before being ligated with AscI adaptors and ligated in intramolecular manner. This results in a pOvaLyso plasmid, containing a unique site upstream of the Ova5′ arm, allowing linearization of the final vector.

For the 3′ arm of the pOvaRH vector, amplification of the downstream region situated between the positions +1657 and +7707 relative to the ATG of the ovalbumin gene with the OvaL 1657 S and OvaL 7707 AS oligonucleotides gives a fragment of size 6050 pb. This fragment was sub-cloned at the EcoRV site of pMCS5 to generate the sense pMCS5-Ova3′ plasmid and antisense pMCS5-Ova3′. Starting with the antisense pMCS5-Ova3′ plasmid, a digestion by HpaI (blunt ends) and NarI (5′ overhang) makes it possible to obtain the 6 kb fragment, that is sub-cloned in pBS-SK at the ApaI (made blunt end) and AccI (compatible with NarI) sites to give the pBS-SK Ova3′ plasmid.

For the 5′ arm of the pLyso RH vector, proximal amplifications with the 1789 S and GE-IN-Ceu-AS lyso oligonucleotides made it possible to isolate a fragment and to sub-clone it directly after treatment with T4 DNA polymerase and phosphorylation by kinase polynucleotide in the pMCS5 commercial vector open in blunt end in EcoRV. This pLyso 5′ fragment contains the partly unpublished 2900 bp sequence of the proximal part of the lysozyme gene as well as the equivalent of the 18 first amino acids of exon 1 corresponding to the peptide signal of the lysozyme. This fragment thus contains a gene splicing donor site as well as the small artificial intron sequence, sequence added by the AS oligonucleotide as described above with the 5′ arm of the pova5′ vector.

For the 3′ arm of the pLysoRHvector, the distal amplifications with the Lyso 2859 S and Lyso 3185 AS oligonucleotides made it possible to isolate a fragment of 2150 bp in 3′ of the gene. These oligonucleotides carry SceI and MluI sites at their end. This fragment submitted to treatment with T4 DNA polymerase, a phosphorylation by the kinase polynucleotide and ligated directly in the pMCS5 plasmid open in blunt end by EcoRV gives the plyso3′ plasmid. Hydrolysis of this plasmid by AfIII/AvrII followed by the action of T4 DNA polymerase and intramolecular ligation makes it possible to generate the pLyso3′ plasmid.

5.3 Construction of the pOvaRH Skeleton

The final assembly of the RH vector through successive additions of different cassettes (selection cassette, insertion of cDNA or gene of interest . . . ) is carried out according to different schema. One schema is described without it being exclusive, other possibilities existing in function of the restriction sites present in certain cDNAs of interest.

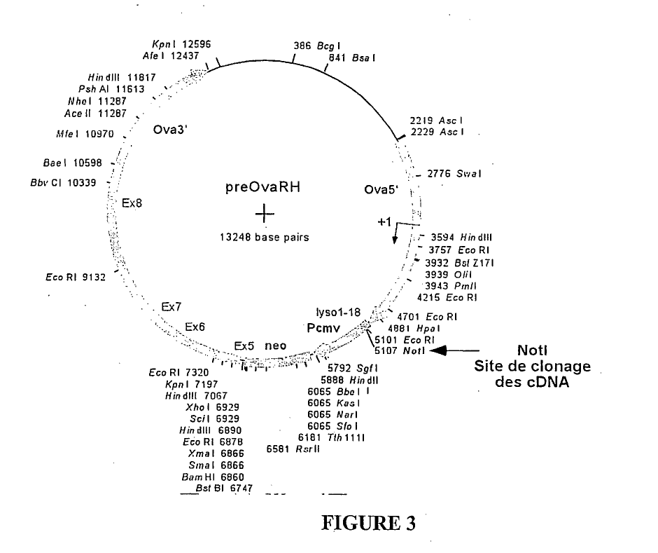

The pCMV NeoOva3′ plasmid is constructed by ligation of the Ova3′ fragment resulting from the purification after cleaving the pOva3′ plasmid by BamHI, with the purified and dephosphoryled pCMV Neo polyA fragment, obtained by BgIII/BamHI hydrolysis of the pCINeo(A-hindIII) plasmid. Several modifications of the polylinker present downstream of the Ova5′lyso insert are also carried out before reassembling the two Ova5′lyso and pCMV-Neo-Ova3′ fragments in a pOvaRH vector (FIG. 3). The NarI/NotI fragment present in pOvaLyso is replaced by an adaptor NotI to give pOvaLyso-NotI, making it possible to conserve the NotI site as unique cloning site for cDNAs of interest by eliminating numerous sites. The pOvaLyso-NotI plasmid is initially cleaved by PacI, then treated by the T4 DNA polymerase to render the DNA blunt end. Release of the OvaLyso-NotI insert is then obtained by a NotI cleaving. The 3 kb OvaLyso PacI^{F}/NotI fragment is cloned at the Kspl sites submitted to the action of the T4 DNA polymerase in order to render the blunt ends/NotI of pCMV-Neo Ova3′. This oriented cloning makes it possible to generate the final pOvaRH vector which presents from 5′ to 3′:
the unique site AseI, allowing linearization of the vector before transfection,
the genomic arm Ova5′lyso (1-18)-intron debut,
the unique site NotI, unique for cloning cDNAs,
the selection cassette with neomycin,
the genomic arm Ova3′.

The pOvaRH vector is then utilizable for inserting different cDNAs of interest.

5.4 Construction of the pLysoRH Skeleton

The Lyso 3′ fragment, obtained by hydrolysis by MluI of the pLyso3′ plasmid is ligated in the plyso5′ plasmid, previously opened and dephosphoryled after hydrolysis by MluI. This results in the pLyso5′Lyso3′ plasmid in which the purified and dephosphoryled pCMV Neo polyA fragment is obtained by NotI/MluI hydrolysis of the pMCS5 CMVNeoPolyA intermediate plasmid. By cloning, the latter is made to flank the selection cassette of the NotI site in 5′, in order to conserve this site for clonings of cDNAs of interest. It is obtained by insertion of the BgIII/BamHI fragment of the pCINeo(Δ-hindIII) plasmid in the open and dephosphoryled pMCS5 in EcoRV. The obtained pLysoRH vector contains successively from 5′ to 3′:
the unique site AseI, that allows linearization of the vector before transfection,
the genomic arm Lyso5′ with the peptide signal 1-18-intron debut,
the unique site NotI, unique for cloning cDNAs,
the selection cassette with neomycin,
the genomic arm Lyso3′.

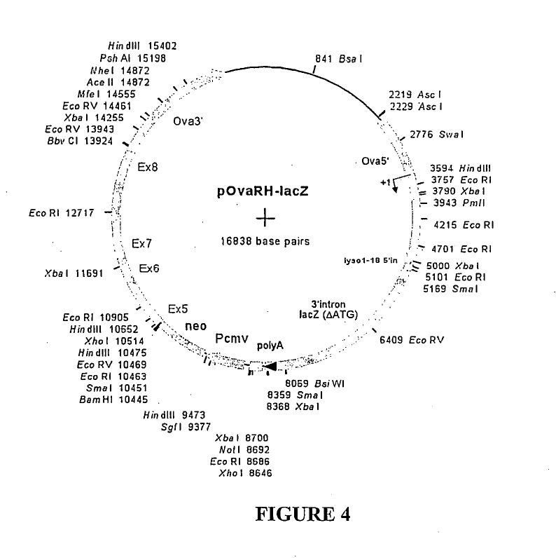

The plysoRH vector is then utilizable for inserting different cDNAs of interest (FIG. 4).

5.5 Application to the Expression of Different Proteins

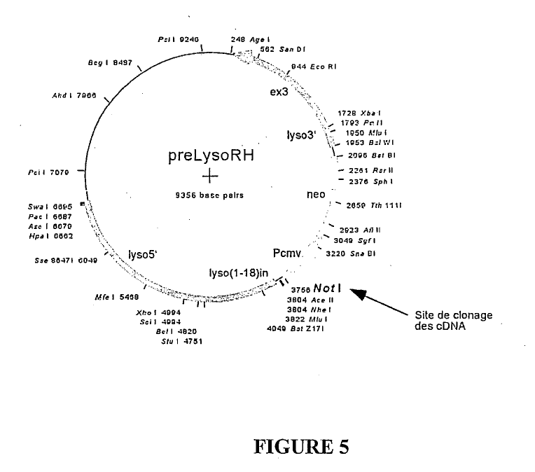

The cDNAs of different proteins of interest or model proteins are inserted in these pOvaRH and pLyso RH vectors. In a non-exhaustive manner, the strategy consists of preparing the cDNA according to the schema described above and inserting it into the open vector in NotI. Variants in the order of clonings of the different parts may be necessary. Partial or total sequencings are carried out in order to verify correct linkage of the different elements inserted. Thus, provided as non-exhaustive examples, vectors were constructed with the cDNAs of pOvaRH Epo erythropoietin, pLysoRH Epo, pOvaRH LacZ beta-galactosidase, pLysoRH LacZ, heliomycin (antibacterial peptide pOvaRH Helio, pLyso RH Helio). FIG. 5 shows pOvaRH LacZ.

A special case is the application of the pOvaRH and pLyso RH vectors to the production of molecules having their own peptide signal. The construction strategy used differs slightly from those described above. For example, the cDNA of interleukin 11 is a nucleotidic fragment of 1100 bp, sub-cloned in the pBS SK vector in the EcoRI sites. Because it is thus unnecessary to fuse the cDNA of interest with the signal peptide of the lysozyme, the cDNA is inserted directly in phase at the level of the ovalbumin ATG, leading to a fusion at the level of the second amino acid of interleukin 11. In this example, the cDNA of IL-11 is submitted to the action of the Taq polymerase in a PCR reaction in the presence of two sense and antisense oligonucleotides presenting the Hind III and XbaI sites. This fragment is thus submitted to HindIII/Xbal double hydrolysis and ligated in the pMCS5 vector, itself prepared open in HindIII/Xbal, after double hydrolysis by these enzymes and dephosphorylation treatment with CIAP. The pMCS5-IL-11 plasmid results from this directional cloning. This plasmid is submitted to HpaI/NsiI digestion, the fragment is purified and ligated in the pOva5′ AS plasmid, itself open in NeoI/PstI, restored as blunt end by the action of Klenow polymerase. This results in the pOvaRH IL-11 plasmid.

This pOvaR5′ IL-11 plasmid is submitted to digestion by NotI, the insert is purified and ligated with the pCMVNeoOva3′ plasmid, itself hydrolysed by NotI and dephosphoryled. This results in the pOvaRH IL-11 plasmid composed of:
the site NotI, (linearization of the vector before transfection),
the genomic arm Ova5′ with the ovalbumin ATG.
the cDNA of IL-11 fused on the ATG and comprising a polyA,
the selection cassette containing the resistant gene to neomycin under dependence of the CMV promoter,
the genomic arm Ova3′.

An analogous strategy makes it possible to obtain the pLysoRH IL-11 vector in the presence of the lysozyme endogenous peptide signal and by deleting the IL-11 peptide signal, and inversely by deleting the endogenous lysozyme peptide signal and supplying the IL-11 peptide signal.

### EXAMPLE 6

### Transfection of Vectors in Different Avian Cells

6.1 Transfection Methods

Two general methods are used for transfection: electroporation and transfection by liposomes. It seems that in the mouse system, electroporation is generally chosen in order to increase the recombination efficiency of the homologous recombination vectors. Nonetheless, few comparative studies have been made.
Electroporation

Electroporation is a method enabling a DNA of very variable size to be entered into a cell by using an electric shock to create a transitory pore at the level of the cellular membrane. Amongst the different parameters capable of influencing the efficiency of this experiment, one can mention the cell concentration (from 1 to 10×10⁶ cells), the conductive solution of the signal containing the DNA cell mixture (PBS, medium without serum . . . ), the concentration of generally linearized DNA (from 5 to 25 μg), the distance of the electrodes (hollows or straight electrodes directly in the culture box or well), the amplitude of the signal depending on the capacitance (from 10 to 1000 μF) and the applied voltage (from 5 to 100 mV for square wave signals and from 200 to 350 V for non-square signals), the number of electric shocks delivered and the form of the electric shock(s) supplied. Form means the way in which a same electric charge is delivered, particularly according to a square or non-square wave signal, inverse exponential . . .

Table 5 illustrates several of these parameters:

TABLE 5
Electroporation efficiency of the pCINeo plasmid
in stem cells

Capacitance (μF)

| | 25 | 150 | 300 | 500 | 700 | 1000 |
|---|---|---|---|---|---|---|
| A Clones | 5 | 122 | 226 | 335 | 408 | 286 |
| B Clones | 9 | 149 | 297 | 454 | 443 | 361 |
| C Clones | 10 | 180 | 218 | 361 | 402 | 365 |
| Average | 8 | 150 | 247 | 383 | 418 | 337 |
| Ecart Type | 3 | 29 | 43 | 63 | 22 | 45 |

Electroporation of the linear pCINeo plasmid is carried out using a BioRad (Gene Pulser II) electroporator. The dissociated stem cells, washed in a medium without serum and adjusted to a concentration of 5×10⁶ cells in 0.8 ml are placed in an electroporation cup (4 mm chamber). 5 to 20 μg of linearized plasmid, prepared in TE, are added to the cells and the mixture is left for 5-10 minutes at ambient temperature, then the electric shock is applied with a fixed capacitance of 560 μF. The cells are placed at 4° C. for about 10 minutes, and then seeded in culture boxes with feeder in the culture conditions for these cells. Generally, the equivalent of a cup, that is 5×10⁶ cells are divided between two 100 mm boxes.

The table shows 3 series obtained with 15 μg of plasmid at 270 V. The clones are enumerated after selection with neomycin for 7 days (250 μg/ml), methanol fixation and Wright/Giemsa colouring.

Liposomes

Liposomes mean any chemical molecule having, among other properties, the characteristics of lipid cations, alone or associated with other elements.

The efficiency of different liposomes has been compared for different cell types. Since the embryonic stem cells are fragile, the presence of serum during transfection is an important element. Different commercial materials were tested. It was shown that the efficiency and toxicity of these components are variable (Pain et al., 1999). Furthermore, the presence of serum makes it possible to reduce the relatively toxic effects of exposure of the cells to lipofectants and thus to conserve the likelihood of obtaining recombinant cells in a satisfactory physiological and morphological state. One advantage among others of the utilization of liposomes is the small number of cells used per test. Table 6 below illustrates a relative transfection efficiency with a simple expression vector:

Table 6: Transfection of Recombination Vectors in Different Cell Types

The cells are seeded in their respective medium with a density of 5×10⁵ to 10⁶ cells per culture box of 100 mm for each cell type. The transfection mixture, varying between 10.5 μg liposomes/3.5 μg DNA to 15 μg liposomes/5 μg DNA per culture box of 100 mm, is put on the cells from 3 to 15 hours according to the case. Neomycin selection is applied at 250 μg/ml and the clones appear 5 to 10 days after transfection. The clones are sampled and analysed by PCR in order to detect the recombination events.

A typical example of amplification is constituted of well cloning in a plate of 24, passage in wells of a 12-well plate, then amplification in a 60 mm or 100 mm culture box. The screening stages set in optimum manner between the passage from the well of a 24-well plate to the well of a plate of a 12-well plate make it possible to limit amplification to only those clones detected positive by PCR. With wells of 6-well plates, 5×10⁵ to 10⁶ cells are usually obtained. Thus only the clones screened positive by PCR are frozen as viable. Amplification for Southern blot analysis also concerns only a small number of clones.

The table brings together different examples obtained with cells of different natures and different passages. It is necessary to note that the transfection and recombination efficiencies seem to be variable according to the vectors and, for a same vector, may be dependent on the physiology (age, state . . . ) of the utilized cells.

TABLE 6

| | | |
|---|---|---|
| | | Clones |
| | Clones | positive |
| Cells | analysed | by PCR |

| (passage nr) | Vectors | nr | nr | % |
|---|---|---|---|---|
| LMH (p25) | pOvaRH Helio | 35 | 1 | 3.8 |
| HD11 | pLysoRH GFP | 40 | 1 | 2.5 |
| HD11 | pLysoRH Epo | 36 | 3 | 8.3 |
| Valo4 (p39) | pOvaRH Helio | 64 | 1 | 1.6 |
| Valo4 (p53) | pOvaRH LacZ | 28 | 1 | 3.6 |
| S86N 48 (p13) | pOvaRH Helio | 48 | 1 | 2.1 |
| S86N 16 (p123) | pOvaRH Helio | 264 | 23 | 8.7 |
| S86N 16 (p129) | pOvaRH LacZ | 168 | 1 | 0.6 |
| S86N 16 (p129) | pOvaRH Helio | 157 | 14 | 8.9 |
| S86N 16 (p137) | pOvaRH Helio | 96 | 3 | 3.1 |
| S86N 16 (p140) | pOvaRH LacZ | 143 | 7 | 4.9 |
| S86N 16 (p203) | pLysoRHx | 94 | 25 | 26 |
| S86N 45 (p71) | pLysoRHx | 94 | 10 | 10.4 |
| S86N 45 (p79) | pLysoRH Helio | 96 | 15 | 15.6 |
| S86N 88 (p14) | pOvaRH LacZ | 79 | 1 | 1.2 |
| Valo8 (p10) | pOvaRH Helio | 35 | 1 | 2.8 |
| Valo8 (p13) | pOvaRH Helio | 120 | 3 | 2.5 |

6.2 Efficiency of Stable Transfection with Recombination Vectors

The efficiency of stable transfection is estimated by the number of clones obtained after selection by the drug detoxified by the gene present in the selection cassette. In the reported cases, neomycin was usually used. Other drugs can be used according to the presence of a different resistance gene in the transfected vector. Among traditional drugs, one finds neomycin, hygromycin, puromycin, phleomycin, zeomycin, blasticidin, viomycin . . . Other selection media can be used like the analogous addition of bases, and traditional selection media such as the HAT medium . . .

The clones are counted at the end of the selection period corresponding to the disappearance of the cells in the experiment's control culture boxes. These controls are either non-transfected cells or cells transfected with a vector not containing a resistance gene to the antibiotic being utilized.

6.3 Cells Utilized

6.3.1 Chicken Embryonic Fibroblasts (CEFs)

Chicken Embryonic Fibroblasts are obtained from the culture of chicken embryos incubated for from 9 to 13 days according to a well established protocol and known to those skilled in the art. These primary cells are amplified and maintained in culture for a small number of passages (usually less than 30) because the cells rapidly show (generally after passages 15 to 20) a progressive stage of proliferation termination and entry into senescence characterized by a lower rate of divisions, and a less and less fibroblast spread morphology. This senescence phase leads to the disappearance of cells except in very rare cases when the spontaneous establishment of avian fibroblast cells, and chicken in particular, was observed (DF-1 line) (Himly et al., 1998; Kim et al, 2001).

The transfections of homologous recombination vectors thus occur during the early passages (from 1 to 10), to optimise both the possibility of selecting and amplifying the selection resistant clones but also to limit an eventual caryotypic derivation and rapid arrival in senescence. Generally, the clones obtained can be isolated, but can only be slightly amplified or not at all. On the other hand, they can be considered as potential sources of modified nuclei for experiments on nuclear cloning.

6.3.2 LMH Line

The LMH cell line is chicken hepatocyte line, obtained by chemical carcinogenesis (Kawaguchi et al., 1987). This line has been characterized at the caryotypic level and shows high polyploidy. Usually, the cells are maintained in DMEM or HamF12, in the presence of 8% foetal calf serum, 2% chicken serum, 10% TPB and antibiotics. These cells represent an interesting dendritic cell type, very different from monocytic cells, fibroblasts and stem cells, and show relative sensitivity to different steroid hormones. Transfections of pOvaRH recombination vectors, particularly in these cells, have made it possible to obtain clones and recombination events. Table 6 illustrates the results of these transfections.

6.3.3 HD11 Line

The HD11 cell line is a line of chicken monocytes immortalized by the presence of the v-myc oncogene brought by an avian retrovirus (Beug et al., 1979). This line is virus productive, able to propagate the oncogene. Certain clones could possibly be non-producers. Generally, the cells are maintained in HamF12 medium, supplemented with 5% foetal calf serum, 2% chicken serum, and 10% TPB. As described in the introduction, the activated macrophages produce quantities of endogenous lysozyme. By modifying them with pLysoRH recombination vectors, these cells can then serve as production model for the molecule inserted into this vector. Thus a knock in production strategy is obtained. Table 6 above illustrates the results of these transfections.

6.3.4 Embryonic Stem Cells

The embryonic chicken stem cells used in the examples are stem cells obtained in vitro from culture of chicken blastoderm cells (Pain et al., 1996; U.S. Pat. No. 6,114,168; WO 96/12793). The cells are maintained, amplified in vitro and above all are capable of proliferating over long time periods. The proliferation profiles emphasize the rapid and slower proliferation phases during the relative establishment of the cells. This particularity of establishment from a primary isolate is completely unique for a cell of avian origin, without external intervention from an immortalizing agent or a chemical transformation process. Thus one can arbitrarily distinguish the early proliferation phases of these cells, observed during the first passages (from passage 1 to passage 20) and the later proliferation phases (passages>20). These stem cells are transfected with different recombination vectors containing the cDNAs of interest.

Tables 6 and 7 illustrate the results obtained with different vectors. It appears that these stem cells are modifiable in different loci by a homologous recombination approach.

Table 7: Transfection of CEC Cells with Different Simple Expression Vectors

5 μg of pcineo vector with 15 μg of Fugene 6 (Roche) is transfected in its circular form on 0-0.8×10⁶ S86N16 cells at different passages. After application of the selection with neomycin by addition of G418 (250 μg/ml) for 5 to 8 days, clones were obtained. The culture boxes are fixed with methanol (100%) and a Giemsa coloration makes it possible to enumerate the clones quickly. In independent tests, this example demonstrates that the cells are stably transfectable with simple vectors, are selectionable and that the clones can be amplified. Nonetheless, the expression levels of the transgenes are very variable from one clone to another, as can easily be visualized by evaluating the fluorescence intensity of the E-GFP marker, for example.

| | Tests | | | | | | | Type |
|---|---|---|---|---|---|---|---|---|
| | 1 | 2 | 3 | 4 | 5 | 6 | Average | spread |
| Number of | 75 | 90 | 135 | 124 | 146 | 106 | 113 | 27 |
| clones | | | | | | | | |

6.4 Physiological State of Cells

By the term physiological state, one understands the state of differentiation, of transcription and of activation of the utilized cells. Modified at the level of the targeted loci by the simple vectors and the homologous recombination vectors, the cells react at the physiological level. Thus it seems useful to follow them in function of their future utilization, the two principals being:
the injection of modified cells into receptor embryos to follow their contribution in the embryos and later at the level of chicks and adult animals,
the production in vitro by the modified cells of the molecules of interest brought into the vectors, particularly but non-exclusively in the case of LMH and HD11 cells.
   6.5 Cloning and Detection of Recombination Events

6.5.1 Cloning

The clones, observed macroscopically and microscopically, are sampled and put directly into a 24-well plate. It is important to take samples of the clones as early as possible in order to limit the effects of mixing between clones. The origin of the clones according to the different culture boxes is mentioned, for example to control the degree of independence of two positive clones. The proliferation conditions of the clones are approximately the same as those used for the parent cells. Once samples have been taken, the clones are observed daily and as soon as the cell density makes it possible, the clones are dissociated and amplified for analysis.

6.5.2 PCR Screening of Clones

The cells of the clones in proliferation in the wells are washed, dissociated and prepared for being reseeded for amplification on the one hand, and for being analysed on the other. The DNA is extracted mainly with the aid of traditional techniques including commercial kits. Different detections are launched to screen the recombination events. The settings for the PCR conditions are determined for each couple of oligonucleotides with DNA extracted from transfected cells, selected and not individually cloned. This ‘pool’ approach makes it possible to obtain material quickly for testing. Usually, a first PCR is carried out to detect the targeted endogenous locus in order to ensure the quality of the extractions and DNAs. A second PCR is carried out to detect a recombination event between an oligonucleotide external to the construction and a sequence common to all the vectors of a same family, for example the lyso 1-18 peptide signal sequence. This PCR is considered as discriminant for the recombination events. New PCR series are obtained on the positive clones in this latter PCR, always with the same external oligonucleotide but with another specific oligonucleotide for each cDNA so as to ensure its presence and the general non-modification of the inserted cDNA. This PCR is considered to be discriminant and enables observation of the positive clones to be reinforced.

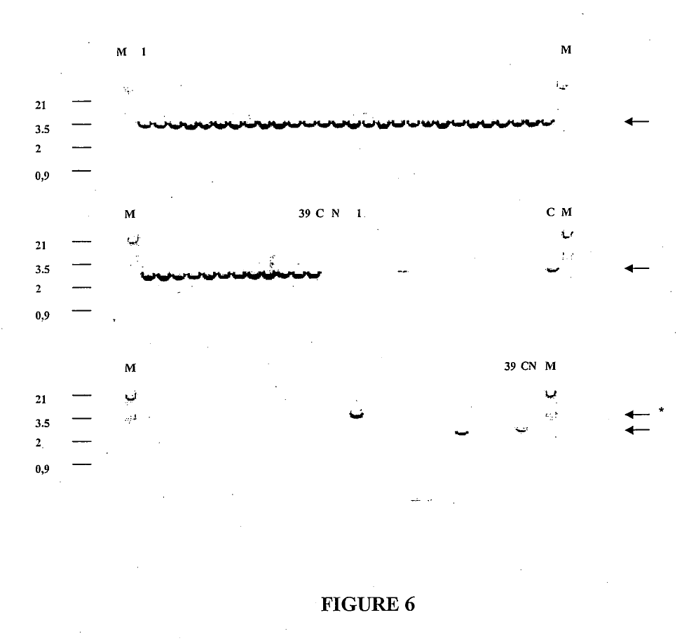

FIG. 6 illustrates this PCR detection of clones. The majority of clones is detected negative, and some are detected recombinant positive. This first sorting means that only a few clones are kept, which are amplified, frozen and analysed by Southern blot.

FIG. 6 Captions: PCR Detection of Recombinant Positive Clones.

As an example, the DNA is extracted with the Promega kit (Madison, WS). The cell packing of each clone is taken in 300 μl lyse buffer (Nuclei Lysis Solution, NLS), in the presence of 20 μg/ml of RNAse. The lysate is homogenised, incubated for 30 minutes at 37° C. and 100 μl of precipitation buffer for proteins (PPS Protein Precipitation Solution) is added. The whole is incubated at 4° C. after vigorous stirring. The supernatant fluid is withdrawn after centrifuging and 400 μl of isopropanol added. After precipitation and centrifuging, the concentrate is washed in ethanol 70° C., dried and taken up in 50 μl of TE (10.1). The volumes can be used proportionally for extractions with more cells. The DNA quantity obtained is estimated by spectrophotometric assay. Extraction of a concentrate equivalent to one well of a 24-well plate gives approximately 0.5 to 2 μg of DNA depending on the cell density in the well. The PCRs are carried out on around 100 to 500 ng.
A first PCR is carried out on 38 samples (Nos. 1 to 39) to detect the endogenous ovalbumin locus and to check the quality of extractions and DNAs. This reaction uses two oligonucleotides, giving an amplified strip of size 2950 bp (tracks 1 to 39, gel upper part).
A second PCR is carried out to detect a recombination event between an external oligonucleotide and the sequence common to all the vectors here, the lyso 1-18 peptide signal sequence. In this case the size expected is 2900 bp (tracks 1 to 39, gel lower part).
M: size marker,
C: DNA control (obtained from a clone mixture),
Cn negative control for the PCR without DNA.

Another PCR is carried out to determine, by exclusion, the sex of the positive clones using specific oligonucleotides from a repeated region of the W chromosome. Only the female clones (ZW) can be detected, the male clones (ZZ) will not be detected.

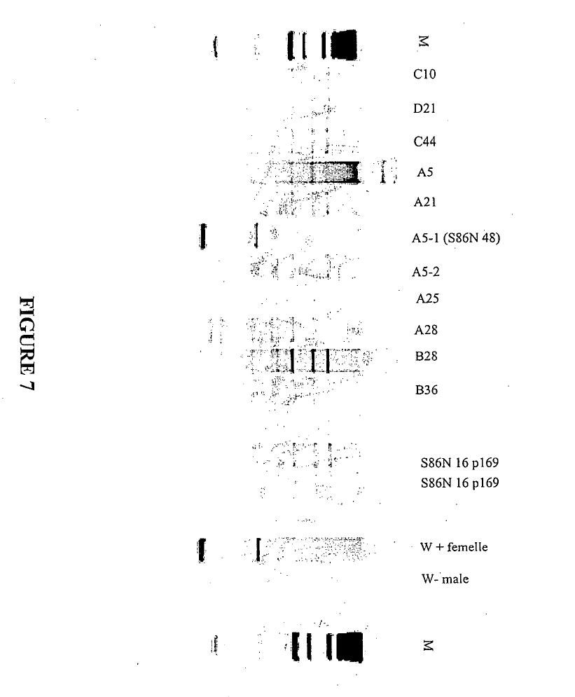

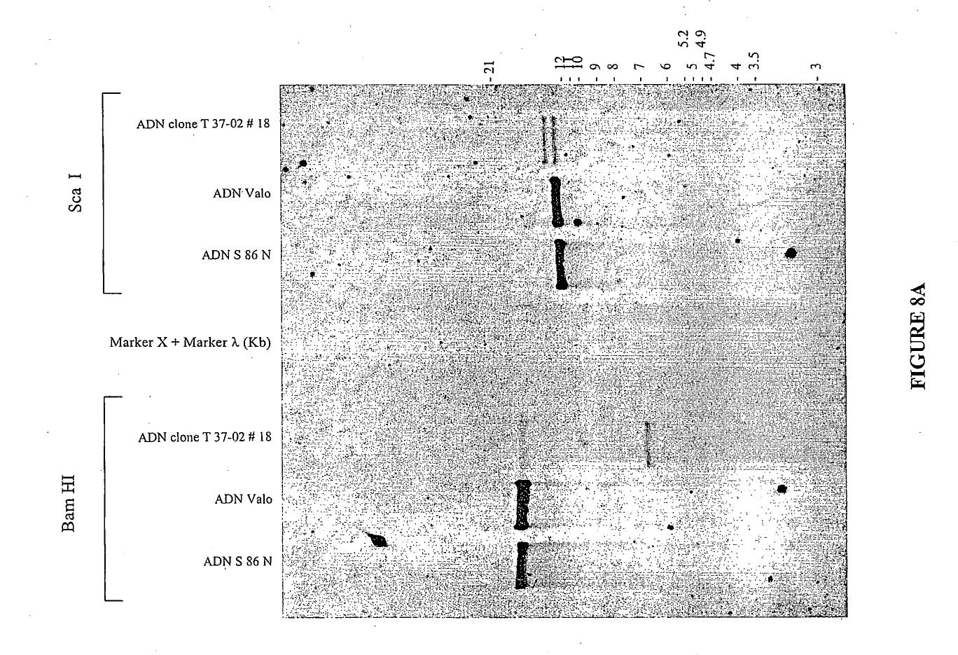

FIG. 7 illustrates this differential detection on several clones.

FIG. 7 caption: Sex Detection of Different Clones Identified Positive by PCR

The DNAs of different clones detected positive by PCR for a homologous recombination event are submitted to a genomic PCR to determine the sex of the clones. The oligonucleotides used are specific to the chicken W chromosome. They can only detect female clones. In the figure, the clones are male in majority, like the utilized cells (S86N16), except for the A5-1 clone, issued from early cells of another origin (S86N48 cells).

Clones obtained with pOvaRH Helio: C10, D21, C44, A5, A21, A5-1

Clones obtained with pOvaRH lacZ: A 5-2, A25, A28, B28, B36

Parent cells: S86N 16

Positive control: DNA of female blood

Negative control: DNA of male blood

M: Marker.

6.5.3 Southern Blot

In order to verify the results obtained in genomic PCR on the positive clones with the two discriminant PCRs for detection of a recombination event, a Southern blot analysis is carried out with the DNA extracted from the clones. This analysis makes it possible to verify that:
the sizes of the targeted locus are preserved and that there is no major deletion,
a single recombination event has taken place and the genome does not contain other random integrations.

The DNA of the clones is extracted according to a usual Proteinase K-SDS method, with phenolic extraction or with the aid of a commercial extraction kit (Qiagen, Promega). In a traditional molecular biology approach, a variable quantity of DNA is submitted to hydrolysis by different enzymes, to make it possible to find the fragments whose size is different in function of the recombination event.

Table 8 illustrates the detection of recombination events by Southern Blot in function of the different probes used.

Table 8: Detection of Recombination Events by Southern Blot

The hydrolysed DNAs are submitted to electrophoretic migration, separating the fragments according to their size. The traditional Southern blot method is applied, with a transfer on an N+hybond membrane (Amersham Pharmacia biotech, UK). The probes used for hybridization are obtained either by PCR or by purification of a plasmidic fraction containing the different genes of interest. For example, the lacZ (1159 bp) and Neo (354 bp) probes are obtained by PCR from plasmidic matrix. The ovalbumin 3′ probe is an intronic probe of 800 bp, obtained from the genomic DNA. The ovalbumin 5′ probe is an intronic probe of 870 bp. These probes are obtained by PCR.

The purified probes are marked with alkaline phosphatase with the aid of the AlkPhos kit (Amersham Pharmacia Biotech, UK) and can be kept for several months at −20° C. For detection, the membranes are pre-hybridized while stirring at 1 ml/cm² in a hybridization buffer and then hybridized at 55° C. by adding the marked probe (5 ng/ml). The membranes are then washed at 65° C. in the primary buffer containing 2M urea, 150 mM NaCl, 50 mM NaPhosphate, 1 mM MgCl2, 0.1% SDS and 0.2% blocking agent, provided by the kit, and then in the secondary buffer containing 50 mM Tris HCL pH 10.0, 100 mM NaCl at ambient temperature.

The chemiluminescence revelation system, ready for use, is added on the washed membrane (1 ml/cm²) and the intensity of the strips is revealed by autoradiography. The size of the detected strips is compared with the expected size.

The criterion for retaining clones is good correspondence between the expected size and that observed. Supplementary hybridizations are carried out (result not shown).

| Digestion | |
|---|---|
| BamH I | ScaI |

| Probe | —H | RH Helio | —RH | RH Helio |
|---|---|---|---|---|
| 3′ Ovalbumin | 18.5 kb | 7 kb | 12 kb | 12.7 kb |
| 5′ Ovalbumin | 18.5 | 9.5 | 12 | 12.7 |
| Neomycin | / | 2 | / | 12 |

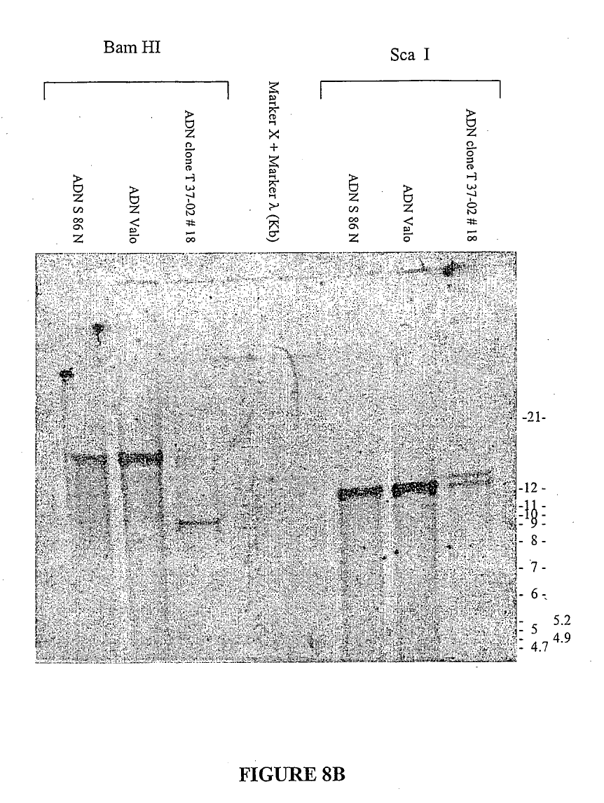

FIG. 8 illustrates this analysis in function of expected sizes.

6.6 Clone Characterization

The different criteria used to characterize parent cells are used to characterize cloned cells.

6.6.1 Karyotype Verification

The karyotype of cells is analysed on the metaphases of obtained cells following a usual protocol after colcemid treatment (pulse of several hours with final 0.05 to 0.15 μg/ml, preferably 0.08 μg/ml) of the proliferating cells. The spread metaphases are observed under a microscope and the number of macrochromosomes counted. The count of minichromosomes is possible but more difficult and a more detailed evaluation by hybridization in situ can also be envisaged. Moreover, FISH verification (fluorescence in situ hybridization) of the presence of the transgene supports the observations made by Southern blot concerning the uniqueness of the event.

6.6.2 Endogenous Phosphatase Alkaline Activity

The first immunocytochemical test used is the detection of endogenous phosphatase alkaline activity. All the clones positive by PCR and validated by Southern Blot analysis are studied in comparison with the parent cells.

6.6.3 Surface Antibodies

Since the parent cells are positive, the presence of specific surface antigens of the stem cells is also sought at the level of the obtained recombinant clones. Reactions with SSEA-1, TEC-01 and EMA-1 antibodies are carried out systematically.

6.6.4 Telomerase Activity

Telomerase activity seems to be one of the best criteria for judging the undifferentiated state of stem cells. Thus, as for the parent cells, the telomerase activity of different recombinant clones is analysed. During the passage of clones for amplification and freezing, a small aliquot of 5×10³ to 5×10⁵ cells is prepared under the form of cellular concentrate and telomerase activity of these cells is evaluated using the ‘Telo TAGGGTélomerase PCR Elisa’ kit (Roche). The level of telomerase activity seems to be a correct reflection of clone quality, especially after the selection pressure applied by the drug.

Table 9 below illustrates several of these characteristics on recombination clones:

TABLE 9
Characterization of pOvaRH Helio clones issuing
from PCR screening

| | AP* | | Antibodies | | Antibodies | | Telomerase | |
|---|---|---|---|---|---|---|---|---|
| | Detection | | TEC01 (%) | | EMA-1 (%) | | 0.07 white | |
| Cells | −RA | +RA | −RA | +RA | −RA | +RA | −RA | +RA |
| S86N16* | ++++ | − | 90 | <10 | 90 | 10 | >1.5 | <0.2 |
| C10 | +++ | − | 70 | <10 | 90 | <10 | 1.23 | 0.282 |
| C44 | +++ | − | 70 | <10 | 90 | <10 | 1.13 | 0.30 |
| D21 | +++ | − | 90 | <10 | 90 | <10 | 1.04 | 0.15 |
| A5-1 | NT | NT | NT | NT | NT | NT | 1.08 | 0.257 |
| A21 | +++ | + | NT | NT | NT | NT | 1.13 | 0.62 |
| A37 | +++ | +/− | NT | NT | NT | NT | 1.09 | 0.566 |

The cells are seeded in the wells of a 12-well plate with 10⁴ cells per well on average. The different characterizations are carried out after fixing in the case of AP and antibody detection and on dry concentrate that is lysed according to the protocol established in the Roche kit in the case of telomerase activity (NT=Non Tested)

Conclusion: as a whole, the clones resemble the parent cells *(S86N16) according to the different markers used to characterize the state of the cells.

6.7 Allelic Conversion

The clones obtained and selected after PCR and Southern blot analysis are a priori modified on a single allele (detection of the wild-type allele in Southern blot analysis). Two modified alleles may be obtained in vivo and in vitro. In vivo, the homozygous animals for modification are obtained after crossing heterozygous animals, themselves descendants of chimeric animals, obtained directly after injection of the modified cells. In vitro, the allelic conversion of the still unmodified second allele is possible in particular by application of a selection of strong doses of the drug G418 (from 0.5 to 5 mg/ml) on the clones. This approach makes it possible to obtain interesting quantities of proteins, both in vitro and in vivo.

### EXAMPLE 7

### In Vitro Test of Recombination Vectors for the Production of Proteins of Interest

The aim of the invention is to produce molecules in the physiological liquid of the animal, particularly the blood and egg white. In order to carry out this approach the vectors, particularly the homologous recombination vectors, are constructed according to the schema described above. These recombination vectors target different loci, principally the ovalbumin locus and that of the lysozyme. An important point is the validation of these vectors by an in vitro approach. In particular it is important to follow the folding of the protein to be produced, the post translational modifications obtained in vitro in an avian system and the influence of the homozygous state of the modified locus on the expression of the molecule of interest. The secretion strategies are also studied via an in vitro model, with different peptide signals and gene splicing strategies according to the constructions.

Two supplementary systems are developed to study each of the main classes of vectors.

7.1 Differentiation In Vitro of Recombined Clones

In a first approach, the differentiation in vitro of stem cell clones can advantageously be used to follow the production of molecules in the supernatant fluid of the cultures. The media conditions used have recourse to the formation of embryoid bodies submitted to the presence of different agents such as inducers, for example dimethylsulphoxide or retinoic acid, TPA, PMA, LPS, factors such as FGF TGFB, MCSF, cMGF, hormones such as the steroid hormones, oestradiaol, dexamethasone, progesterone or other hormone families.

7.2 Stimulation of Recombined Line Cells

7.2.1 Validation of pOvaRH Vectors.

The LMH cells are avian cells issuing from a hepatic tumour. According to several examples taken from published literature, the ovalbumin gene seems able to be activated in the LMH line under different conditions of hormonal stimulations. In particular, a combination of insulin, oestradiol and dexamethasone (steroid mimicking the action of glucocorticoids) enables fairly significant induction of the ovalbumin gene. This stimulation can be used on the recombined clones of LMH cells. Co-transfections of the ER and COUTF nuclear receptors are also envisaged to raise the level of these receptors in the cells and thus allow better induction of the ovalbumin gene under the effect of the different inductors utilized. The vectors of the pOvaRH family are used preferentially in this approach.

7.2.2 Validation of the pLyso RH Vectors

It seems possible to induce the differentiation of HDl1 cells, chicken monocytes line in macrophage cells by different treatments, including action by lipopolysaccharides (Goethe et al., 1994; Goethe and Phi-Van, 1998). In a stimulation approach by these substances, the supernatant fluid of the recombined clones, particularly with the vectors of the pLysoRH family, is studied and makes it possible to obtain significant quantities of proteins of interest.

Table 10 below illustrates this production of molecules of interest starting from HDll cells recombined with one of the vectors of the pLysoRH family specifically under inducer action including the LPSs.

Table 10: Stimulation of Transfected HD11 Cells with the pLysoRH Epo Vector

The HD11 cells are transfected and recombined clones obtained with the pLysoRH Epo vector. Two analyses are carried out on the clones stimulated by the LPS (5 μg/ml):
A: an RT-PCR reaction to detect the messengers of the endogenous lysozyme in the induced non-transfected cells and the messengers of erythropoietin in the clones of recombined cells.

B: Detection by Elisa reaction (Roche kit) of the supernatent fluid of stimulated or non-stimulated clones makes it possible to titer the presence of erythropoietin.

| | | D.O. to 450 | Concentration |
|---|---|---|---|
| Standard | S1 | 0 | 0 |
| | S2 | 0.12 | 11 |
| | S3 | 0.26 | 20 |
| | S4 | 0.46 | 41 |
| | S5 | 0.86 | 86 |
| | S6 | 2.00 | 179 |
| Positive control | | 0.79 | 71.4 |
| Negative control | | 0.09 | 1.2 |
| Parental HD11 | −LPS | 0 | <0.5 |
| Non-recombined | +LPS | 0 | <0.5 |
| clones | | | |
| Clone 1 pLysoRH Epo | −LPS | 0.67 | 61.3 |
| | +LPS | 2.22 | 199 |

Conclusion: Clone 1 produces large quantities of erythropoietin in the culture supernatant fluid.

### EXAMPLE 8

### Injection of Modified Cells into Receptor Embryos and Animal Obtainment

The clones of modified stem cells are characterized as parent cells using the different immunocytochemistry markers described above. After these controls, certain clones are used to generate modified animals, intended to produce the molecule of interest. Among the different strategies used to produce these animals, one can distinguish:

8.1 Intrablastoderm Injection

The modified cells are injected into the blastoderm cavity of a receptor embryo in blastula stage according to a protocol described above (Pain et al., 1996, Patents U.S. Pat. No. 6,114,168; WO 96/12793) The embryos used in this approach are non-incubated or just incubated. The cells will take part in the colonisation of the embryos. Different supplementary methods make it possible to evaluate the contribution level of the injected cells; for example, one method is based on a phenotype difference between a non-pigmented white receptor strain and a coloured donor strain (from which the utilized cells are provided). Another method is the follow-up of the presence of the transgene by genomic PCR using specific oligonucleotides.

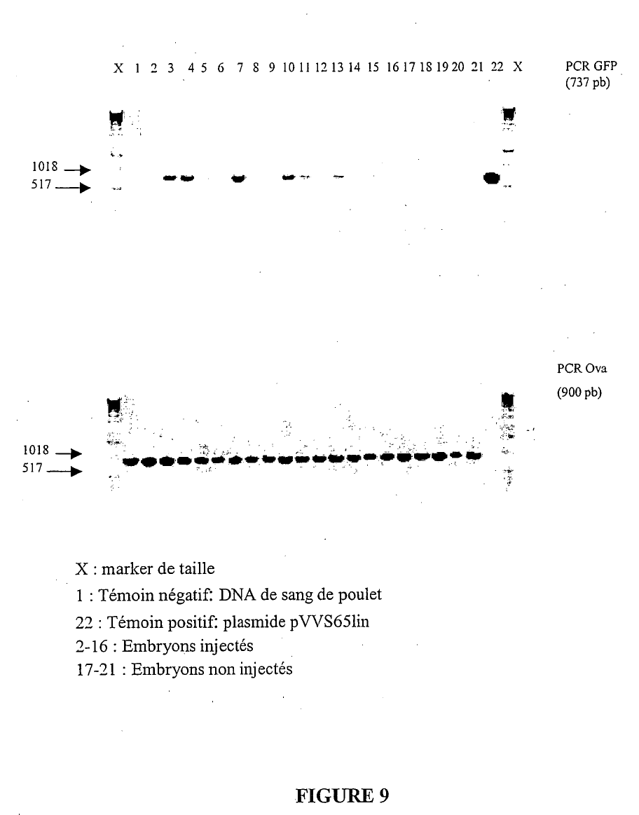

FIG. 9 shows the phenotype contribution of a clone modified with a simple expression vector.

FIG. 9 caption: Obtainment of Chimera with the Modified Clones

Chimera obtained with modified clones by the simple vector pEGFB-SV40 Neo.

The eggs injected by blastodermic path are opened at different stages and the embryos taken out for analysis. The extracted DNA is submitted to a genomic PCR reaction with different oligonucleotides to detect the presence of the transgene (E-GFP here) The upper part of the gel thus shows 21 analysed samples, 11 of them detected positive out of the 15 injected embryos analysed. The lower part shows the analysis carried out with another pair of oligonucleotides, making it possible to validate the DNA and extraction quality.

Conclusion: the embryonic cells modified by a simple expression vector can contribute and colonise the embryos in an efficient manner.

Table 11 below illustrates chimera obtainment with a recombined clone.

Table 11: Different Phenotype Chimera Obtained with a Recombined Clone by the pOvaRH Helio Vector, Injected by Intra-Blastoderm Path. The Apparent Chimeric Level is Variable.

| Injected | | | |
|---|---|---|---|
| cells | Vector | Live animals | Chimera (%) |
| S86N 66 (p5)* | x | 13 | 1 (8%) |
| S86N 48 clone | pOvaRH Helio | 37 | 1 (8%) |
| A5 | | | |
| S86N 48 clone | pOvaRH Helio | 43 | 3 (7%)** |
| A5 | | | |

*Non-modified cells in control
**6 other chimeric animals were obtained in embryonic state.

The DNA of the live chimeric animals was extracted from a feather biopsy and a PCR detection confirmed the presence of the transgene in the samples (result not shown).

8.2 Intra-Cardiac Injection

The modified cells are injected into the blood circulation via the intra-cardiac path of an embryo incubated for from 48 to 72 hr. At this stage of development, the morphogenesis of the embryo allows significant accessibility to the cardiac cavity in formation. The cells are injected directly, using fine capillaries. Development continues normally until hatching.

Table 12 below illustrates the phenotype contribution of modified clones in embryo tissues by intra-cardiac injection.

Table 12: Obtainment of Phenotype Chimera by Intra-Cardiac Injection

The cells of a clone modified with a simple vector (in the example clone 14A is obtained with the pVVS65 vector containing pCMV E-GFP SV40 Neo) or a recombination vector are directly introduced by intra-cardiac injection into the circulation of an embryo incubated for several hours (from 36 to 72 hours). In the example, the tissues are sampled on embryos of from 5 to 21 days depending on the case. A genomic PCR makes it possible to detect the presence of the transgene, while another PCR on an endogenous gene validates the quality of the analysed DNA.

| | Injected | Analysed | Positive | |
|---|---|---|---|---|
| Cells | embryos | embryos | embryos | % |
| Exp 1 | 40 | 16 | 2 | 12.5 |
| Exp 2 | 40 | 18 | 3 | 16.7 |
| Exp 3* | 50 | 14 | 2 | 14.3 |
| Exp 4* | 50 | 18 | 2 | 11.1 |
| Exp 5* | 50 | 22 | 1 | 4.5 |

*different tissues are sampled on chicks when hatching, and analysed. PCR detections are in general positive on several different tissues.

8.3 Utilization of Modified Nuclei as Nuclei Source in a Nuclear Transfer Process.

The modified cells are sources of modified nuclei that can be used in nuclear transfer techniques in a receptor ovocyte in order to reconstitute an embryo. In this case, the modification supplied by the nucleus is carried directly by the animal in its genetic heredity and therefore transmitted to all its descendents. Present fusion techniques for the cell and/or the nucleus alone with the prepared ovocyte make it possible to envisage such an approach. The activation phases then allow efficient reprogramming of the donor nucleus in place of the haploid genetic material of the receptor ovocyte.

### REFERENCES

1. Utilization of an avian cell for the production of an exogenous protein of interest in an animal belonging to the avian species, characterized in that said cell is transformed by an expression vector comprising the coding gene for said protein, said cell being introduced either in the sub-germinal cavity of an embryo, the blood circulation of the embryo, or acting as a nucleus source for nuclear transfer in an ovocyte, enucleated or not, or in which the chromosomes have been destroyed.

2. Utilization according to claim 1, characterized in that the vector allows a tissue-specific expression, in particular in the oviduct, the liver, the blood, the bone marrow and the lymphoid organs.

3. Utilization according to claim 1 or claim 2, characterized in that the vector is a homologous recombination vector possessing 5′ and 3′ arms for homology with the sequences of a given locus.

4. Utilization according to claim 1, characterized in that the targeted locus is selected from among the locus of ovalbumin, ovomucoids, conalbumin and lysozyme.

5. Utilization according to claim 1, characterized in that the homologous recombination vector comprises a linkage in a plasmidic base of at least one element taken successively from amongst:
a) a genomic DNA fragment containing the 5′ homologous arm of the targeted gene fused with,
b) a secretion signal nucleotide sequence fused with,
c) a short intronic nucleotide sequence fused with,
d) the coding nucleotide sequence for the protein of interest fused with,
e) a termination poly A transcription sequence fused with,
f) a positive selection cassette comprising a promoter, a gene for resistance to a selection agent and a poly A sequence for transcription termination, said cassette being able to be fused with,
g) a genomic DNA fragment containing the 3′ homologous arm of the targeted gene,
h) a negative selection cassette comprising a promoter, a gene ensuring the transformation of a substrate present in the culture medium into a toxic substance for the gene expression cell and a poly A sequence for transcription termination.

6. Utilization according to claim 1, characterized in that the vector comprises d) the coding sequence for the fused exogenous protein at its 5′ end with c) a short intronic sequence comprising in particular the sequence SEQ ID No 1 itself fused with e) a secretion peptide signal sequence, in particular the coding sequence for the peptide signal of the lysozyme comprising the SEQ ID No 2 sequence.

7. Utilization according to claim 1, characterized in that the vector comprises d) the coding sequence for the exogenous protein fused at its 3′ end with a poly A sequence.

8. Utilization according to claim 1, characterized in that the vector comprises at least one IRES sequence in fusion with at least two coding sequences for the exogenous protein of interest.

9. Utilization according to claim 1, characterized in that the vector comprises at least one IRES sequence in fusion with at least two coding sequences for different chains constituting a protein of interest, in particular the light and heavy chains of an antibody of any nature whatsoever, in particular a monoclonal antibody, a fab fragment.

10. Utilization according to claim 1, characterized in that the IRES sequence is taken in the group of IRES sequences of group I or group II, in particular the V130, Idemfix, Zam sequences.

11. Utilization according to claim 1, characterized in that the vector is an expression vector comprising the coding sequence for the protein of interest fused with at least one element taken from amongst:
a) a promoter, selected in particular from amongst the promoters of genes of ovalbumin, ovomucoids, conalbumin and lysozyme,
b) a peptide signal sequence,
c) a nucleotide poly A sequence for transcription termination.

12. Utilization according to claim 1, characterized in that the expression vector comprises an IRES sequence fused with at least two coding sequences for the same protein of interest or for different coding sequences.

13. Utilization according to claim 1, characterized in that the cell is a primary embryonic avian cell.

14. Utilization according to claim 1, characterized in that the cell is an embryonicavian stem cell, in particular the embryonic stem cells resulting from the culture of blastoderms.

15. Utilization according to claim 1, characterized in that the avian embryonic cell is of a positive phosphatase alkaline phenotype, in particular a positive phosphatase alkaline embryonic stem cell phenotype.

16. Utilization according to claim 1, characterized in that the avian embryonic cell reacts specifically with at least one antibody selected from amongst ECMA-7, SSEA-1, SSEA-3, TEC-O1, EMA-1 and EMA-6, in particular an embryonic stem cell, an embryonic germ cell.

17. Utilization according to claim 1, characterized in that the cell is a primary avian cell of phenotype defined in particular for a primary fibroblast, an epithelial cell, an endothelial cell.

18. Utilization according to claim 1, characterized in that the cell is an avian cell derived from a primary embryonic cell and spontaneously established by culture or with the aid of different immortalizing agents, in particular the cells derived from embryonic stem cells induced to differentiate under the action of different inducer agents, in particular retinoic acid, dimethylsulphoxide, TPA or specific culture conditions, in particular by forming embryonic bodies.

19. Utilization according to claim 1, characterized in that the cell is an established avian cell line, in particular the LMH hepatic cells, the HD11 monocyte cells, the QT6 fibroblast cells.

20. Utilization according claim 13, characterized in that said cell is transformed with an expression vector expressing a protein of the Rad family, in particular the Rad54 protein.

21. Method for obtaining an avian cell modified by one of the vectors defined according to claim 5.

22. Method for obtaining an avian cell modified according to claim 21, characterized in that it comprises the following stages:
a) introduction of the defined vector according to claim 5 in an avian embryonic cell by a transfection method, in particular with the aid of a liposome, a polycation or by electroporation,
b) selection of cells by addition of a selection agent in the culture medium, in particular geneticin in a concentration range from 100 to 500 μg/ml,
c) screening of resistant clones and amplification.

23. Method according to claim 21, characterized in that the recombination vector targets the lysozyme locus.

24. Method according to claim 21, characterized in that the culture supernatant fluid from recombined clones contains the exogenous protein of interest, in particular after induction of the clone with the aid of different inducers, in particular retinoic acid, dimethylsulphoxide, TPA or specific culture conditions, in particular by forming embryonic bodies.

25. Method according to claim 21, characterized in that the two alleles of the targeted locus are modified.

26. Method according to claim 21, characterized in that the cells are primary embryonic avian cells.

27. Method according to claim 21, characterized in that the cells are embryonic avian stem cells, in particular embryonic stem cells resulting from the culture of blastoderms.

28. Method according to claim 21, characterized in that the cells are cells showing a positive phosphatase alkaline phenotype, in particular a positive phosphatase alkaline embryonic stem cell phenotype.

29. Method according to claim 21, characterized in that the avian embryonic cells react specifically with at least one antibody selected from amongst ECMA-7, SSEA-1, SSEA-3, TEC-01, EMA-1 and EMA-6.

30. Method according to claim 21, characterized in that the cells are primary avian cells of phenotype defined in particular for a primary fibroblast, an epithelial cell, or an endothelial cell.

31. Method according to claim 21, characterized in that the cells are avian cells derived from a primary embryonic cell and established in line with the aid of different immortalizing agents, in particular derived cells from embryonic stem cells induced to differentiate under the action of different inducer agents in particular retinoic acid, dimethylsulphoxide, TPA or specific culture conditions, in particular by forming embryonic bodies.

32. Method according to claim 21, characterized in that the cells are established aviancell lines, in particular the LMH hepatic cells, the HD11 monocyte cells, the QT6 fibroblast cells.

33. Method according to claim 21, characterized in that said cells are transformed with an expression vector expressing a protein of the Rad family, in particular the Rad54 protein.

34. Method according to claim 21, characterized in that the medium used comprises antiretinoic acid antibodies (ARMA).

35. Method according to claim 21, characterized in that the utilized medium comprises a cytokine chosen from amongst the group constituted by LIF, IL-11, IL-6 and their different mixtures.

36. Method according to claim 21, characterized in that the used medium comprises different factors, in particular SCF, IGF-1, bFGF, CNTP and Oncostatin.

37. Method for obtaining an animal belonging to the avian species capable of expressing an exogenous protein of interest, characterized in that it comprises the following stages:
a) obtainment of avian cells modified by the method defined according to claim 21,
b) introduction of the cell obtained in stage a) into the sub-germinal cavity of an embryo, in the blood circulation or by nuclear transfer of the nucleus of said cell to an enucleated or non-enucleated ovocyte, and
c) incubation of the embryo obtained in stage c).

38. Method according to claim 37, characterized in that the vector used in stage a) enables a tissue specific expression, in particular in the oviduct, the liver, the blood, the bone marrow and the lymphoid organs.

39. Method according to claim 37 to obtain an animal belonging to the avian species with a tissue-specific expression of an exogenous protein of interest, characterized in that the vector is a homologous recombination vector possessing, among different constitutive elements necessary for its functioning, 5′ and 3′ arms homologous with the sequences of a given locus, especially a locus selected from amongst the ovalbumin, ovomucoids, conalbumin and lysozyme locus.

40. Method according to claim 37, characterized in that the vector comprises the coding sequence for the fused exogenous protein with at least one element selected from amongst an intronic sequence, a secretion peptide signal sequence, in particular the peptide signal of the lysozyme comprising the SEQ ID No 2 sequence, a poly A sequence, an IRES and a promoter, chosen in particular from amongst the promoters of the genes of ovalbumin, ovomucoids, conalbumin and lysozyme.

41. Method according to claim 37, characterized in that stage b) furthermore comprises the transformation of avian cells with a vector expressing a protein of the Rad family, in particular RadS4.

42. Method for production of a protein of interest comprising the extraction of the exogenous protein expressed in the tissues of an animal obtained from the method according to claim 37.

43. Method for production of a protein of interest comprising the extraction of the exogenous protein expressed in the supernatant fluid of the cells issuing from the method according to claim 21.

44. Method according to claim 42, characterized in that the protein is extracted from the blood, the yolk or the white of an egg.

45. Animal belonging to an avian species able to be obtained from the method according to claim 37, characterized in that it expresses an exogenous protein in a specific tissue.

46. Animal according to claim 45, characterized in that it expresses an exogenous protein in the liver, the blood, bone marrow, the lymphoid organs or the oviduct.

47. Egg able to be obtained starting from an animal according to claim 45, characterized in that part of these components, in particular the ovalbumin, ovomucoids, conalbumin and lysozyme are partly or totally replaced by an exogenous protein of interest, selected in particular from amongst the peptides of therapeutic interest, the interleukins, the cytokines, hormones and antibodies.

48. Egg able to be obtained starting from an animal according to claim 47, characterized in that it comprises a proportion of exogenous protein comprised between several mg (1 to 10 mg) and 500 mg of dry material instead of and in place of a part or the totality of at least one endogenous protein, chosen in particular from amongst ovalbumin, ovomucoids, conalbumin, lysozyme and avidin.


SEQUENCE LISTING (17 sequences)

SEQ ID NO: 1 - DNA, 161 bp, Artificial sequence
  Description of artificial sequence Sequence of artificial intron of pOvaRH and pLysoRH vectors
gtaagtatcaaggttacaagaattcgcggccgctctagaactagtggatcccccgggctgcaggaattcgatatcaagcttataacttcgtataatgtatgctatacgaagttatcccatcattggtcttactgacatccactttgcctttctctccacag

SEQ ID NO: 2 - DNA, 54 bp, Artificial sequence
  Description of artificial sequence Lysozyme secreting signal peptide
atgaggtctttgctaatcttggtgctttgcttcctgccccttgctgctcttggt

SEQ ID NO: 3 - protein, 18 aa, Artificial sequence
  Description of artificial sequence Lysozyme secreting signal peptide
MRSLLILVLCFLPLAALG

SEQ ID NO: 4 - DNA, 34 bp, Artificial sequence
  Description of artificial sequence cDNA-SmaI AS oligonucleotide
atacccggnnnnnnnnnnnnnnnnnnnnnnnnnn

SEQ ID NO: 5 - DNA, 52 bp, Artificial sequence
  Description of artificial sequence GE-Int-AS oligonucleotide
aaccttgatacttactccaagagcagcaaggggcaggaagcaaagcaccaag

SEQ ID NO: 6 - DNA, 82 bp, Artificial sequence
  Description of artificial sequence GE-Int-Ceu AS oligonucleotide
tcgctaccttaggaccgttatagttattgtaaccttgatacttactccaagagcagcaaggggcaggaagcaaagcaccaag

SEQ ID NO: 7 - DNA, 30 bp, Artificial sequence
  Description of artificial sequence IL-11 (-ATG) S oligonucleotide
ctgaagcttgttaactgtgtttgccgcctg

SEQ ID NO: 8 - DNA, 30 bp, Artificial sequence
  Description of artificial sequence IL-11 3′ncRevAS oligonucleotide
cgctctagatgtatgacacatttaattccc

SEQ ID NO: 9 - DNA, 33 bp, Artificial sequence
  Description of artificial sequence Lyso 1-18 S oligonucleotide
ggggaattccatgggtatgaggtctttgctaat

SEQ ID NO: 10 - DNA, 30 bp, Artificial sequence
  Description of artificial sequence Lyso 1789 S oligonucleotide
actatgactactggcaggagaatgaggaac

SEQ ID NO: 11 - DNA, 28 bp, Artificial sequence
  Description of artificial sequence Lyso 2859 S oligonucleotide
acttgcacacggaatatggaagggcatt

SEQ ID NO: 12 - DNA, 33 bp, Artificial sequence
  Description of artificial sequence Lyso 3185 AS oligonucleotide
gtttgcgtatagtcgttttaatgagggatgcgt

SEQ ID NO: 13 - DNA, 40 bp, Artificial sequence
  Description of artificial sequence OvaL 1657 S oligonucleotide
cgcgtctcgagccagagagctcatcaattcctgggtagaa

SEQ ID NO: 14 - DNA, 40 bp, Artificial sequence
  Description of artificial sequence OvaL 7707 AS oligonucleotide
cgcgtctcgagttctatatggggtgtggtgacagagcaat

SEQ ID NO: 15 - DNA, 39 bp, Artificial sequence
  Description of artificial sequence OvaL 80 AS oligonucleotide
aattcggatcccatgatggcaatggggcagtagaagatg

SEQ ID NO: 16 - DNA, 40 bp, Artificial sequence
  Description of artificial sequence Oval-2995 S oligonucleotide
aattcggatccaacatttactgggaagcacatctatcatc

SEQ ID NO: 17 - DNA, 57 bp, Artificial sequence
  Description of artificial sequence SmaI3′ in cDNA S oligonucleotide
atacccgggattggtcttactgacatccactttgcctttctctccacagnnnnnnnn